(19) United States
(12) Patent Application Publication
(10) Pub. No.: US 2025/0352656 A1
(43) Pub. Date: Nov. 20, 2025

(54) SYNTHETIC CATIONIC PEPTIDOMIMETICS, THEIR DERIVATIVES AND PREPARATION THEREOF ALONG WITH COMPOSITIONS FOR VARIOUS MEDICAL APPLICATIONS

(71) Applicant: PEPTOMER THERAPEUTICS PRIVATE LIMITED, Faridabad (IN)

(72) Inventors: Kantaraja CHINDERA, Karnataka (IN); Manohar MAHATO, New Delhi (IN); Sahil CHAHAL, Sonipat (IN); Anupam MISHRA, Prayagraj (IN); Nikita VERMA, Faridabad (IN); Princy DAHIYA, Sonipat (IN)

(21) Appl. No.: 18/859,237

(22) PCT Filed: Apr. 24, 2023
(86) PCT No.: PCT/IN2023/050399

§ 371 Date: Oct. 23, 2024

(30) Foreign Application Priority Data
Apr. 24, 2022 (IN) 202211010038

(51) Int. Cl. A61K 47/64; C07C 335/28; C07K 7/02; C08G 69/08

(52) U.S. Cl. CPC A61K 47/6455; C07C 335/28; C07K 7/02; C08G 69/08; C07C 2603/18

(57) ABSTRACT

The present invention discloses cationic guanidine based peptides, oligomers, and oligomer-peptide hybrid compounds, and also envisages within its scope monomers for obtaining peptides, oligomers and oligomer-peptide hybrid compounds, and the processes thereof. The present invention additionally relates to the composition for administration of the compounds and their utility as antimicrobial, antifungal agent and as a carrier therapeutic molecules for drug delivery applications.

### FIELD OF THE INVENTION

The present invention is generally related to the field of chemistry. In particular, the present invention is related to novel thiourea monomers capable of yielding oligomers, peptides and peptide-oligomers with cationic group/s in the backbone, process for preparation of the homo- and hetero-oligomers and peptides, compositions containing these polymers and uses thereof.

### BACKGROUND OF THE INVENTION

Polymeric guanidines are cationic amphipathic polymers which are reported to have biological activity and therefore used in pharmaceuticals. Chemically these polymers have cationic guanidine groups connected by a suitable aliphatic linker; they bear a net positive charge at physiological pH. Several derivatives of polymeric guanidine have been synthesized for antimicrobial applications. Among these polyhexamethylene guanidine (PHMG) and poly-[2-(2-ethoxy)-ethoxyethyl)-guanidinium-chloride] (PEEG) are widely used. PHMG bears a repeating unit of a guanidine moiety connected by a hexamethylene linker and has broad spectrum antimicrobial activity. Current method of their synthesis creates many side reactions and impurities, thus limiting their in-vivo use as antimicrobials.

While there are certain cationic polypeptides available in the market, they are not prevalent in usage. Further some of the oligomers and the peptides found in the art are not efficacious. Hence, there is a need for novel oligomers, peptides and peptide-oligomers with cationic group/s in the backbone, which are efficacious and provide alternative pharmaceutical options to the prior art compounds.

### OBJECT OF THE INVENTION

An object of the present invention is to provide novel monomers, and their use in preparing homo and hetero peptides, oligomers, oligomer-peptide hybrids, with at least one guanidine moiety in the backbone, a process of producing the monomer and the peptide, oligomer, oligomer-peptide hybrid, a pharmaceutical composition comprising the said peptide or oligomer or oligomer-peptide hybrid and uses thereof.

### SUMMARY OF THE INVENTION

The present invention discloses novel monomers compatible with synthesis of unnatural peptide, oligomer or peptide-oligomer hybrid sequence. It also discloses a process of producing the peptide, oligomer, oligomer-peptide hybrid compounds, and their uses thereof. The cationic guanidine group is formed during the synthesis on a solid support in the backbone of peptides, oligomers, oligomer-peptides during synthesis. The present invention also provides utility of the compounds of the present invention in antimicrobial activity and as carrier for other therapeutic molecules for drug delivery applications.

### BRIEF DESCRIPTION OF THE DRAWINGS

FIG. 1 depicts uptake of peptides into mammalian cells

FIG. 2 depicts peptide-CPP uptake into Vero cells

FIG. 3 depicts peptide uptake into yeast cells

FIG. 4 depicts interaction of peptides with oligonucleotides and pDNA

FIG. 5 depicts delivery of oligonucleotides into mammalian cells

FIG. 6 depicts delivery of pDNA into mammalian cells

FIG. 7 depicts delivery of proteins into mammalian cells

FIG. 8 depicts antibacterial activity of compounds against Escherichia coli and Staphylococcus aureus

FIG. 9 depicts potentiation of antibiotics against Staphylococcus aureus

FIG. 10 depicts antifungal activity of peptides against Candida albicans

### DETAILED DESCRIPTION OF THE INVENTION

a. Compounds of the Present Invention

### Formula 1: General Formula for Monomers

The present invention discloses novel monomers (Formula 1) highly compatible for synthesis of peptide, oligomer, oligomer-peptide hybrid compounds.

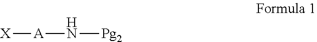

where X is defined with the general formula as

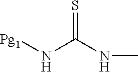

and A is selected from the group consisting of -L-,

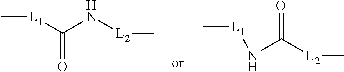

wherein the linker groups, “L, L1, L2,” are defined in Table 1,

The linker groups “L, L1, L2, are an aliphatic group containing propyl (C₃), butyl (C₄), hexyl (C₆), N-substituted alkyl amide, C₁-C₁₄₀ carbon atoms, selected from group comprising an alkyl group, such as, methylene, ethylene, propylene, C₄, C₅, C₆, C₇, Cs, C₉ or C₁₀; C₁-C₁₀, —C₂₀, —C₃₀, —C₄₀, —C₅₀-C₆₀, —C₇₀, —C₈₀, —C₉₀, —C₁₀₀, —C₁₁₀, —C₁₂₀, —C₁₃₀ or —C₁₄₀, alkyl; cycloaliphatic, heterocyclic, aromatic, aryl, alkylaryl, arylalkyl, oxyalkylene radicals; a polyalkylene radical optionally interrupted by one or more, preferably one, oxygen, nitrogen or sulphur atoms, functional groups, saturated or unsaturated cyclic moiety; or polyethers selected from the group comprising PEG/derivatives of PEG/analogues of PEG. Further, these linker groups comprise additional cationic moieties selected from the group comprising secondary or tertiary or quaternary amines, or guanidine, or aminoguanidine, diaminoguanidine or analogue, derivative cationic groups either in the backbone of the linker or side chain of the linker. The linker may additionally comprise an amide bond.

Pg1 is a protecting group/s attached to the thiourea in the monomer, it is chosen in such a way that it remains attached to the resulting guanidine moiety in the oligomer through the synthesis process. Pg1 is only removed during final cleavage of the oligomer from the resin;

Pg2 is a protecting group attached to terminal amine (NH) to facilitate step-by-step addition of oligomers to a growing oligomer chain during solution/solid phase synthesis;
wherein Pg1 or Pg2 is independently an orthogonal protecting group selected from the group consisting of Pg1 as Fmoc/Cbz or Pg1 as Boc/Cbz and Pg2 as Boc, Pg2 as Fmoc.

The examples of compounds having Formula 1 with substitutions X, L, Pg₂ are shown in Table 1.

TABLE 1
Examples of compounds with substitutions X, L, Pg₂ in Formula 1

| | | | |
|---|---|---|---|
| IUPAC Name | X | L | Pg₂ |

| | |
|---|---|
| N-(9-Fluorenyl methoxy carbonyl)- | Boc |
| N′-(6-tertbutoxycarbonyl | |
| aminohexylene) thiourea | |
| N-(9-Fluorenyl methoxy carbonyl)- | Boc |
| N′-(4-tertbutoxycarbonyl amino | |
| butylene) thiourea | |
| N-(-Fluorenyl methoxy carbonyl)- | Boc |
| N′-(3-tertbutoxycarbonyl amino | |
| propylene) thiourea | |
| N-(Benzyloxy carbonyl)-N′-(6- | Boc |
| tertbutoxycarbonyl aminohexylene) | |
| thiourea | |
| N-(Benzyloxy carbonyl)-N′-(4- | Boc |
| tertbutoxycarbonyl amino | |
| butylene) thiourea | |
| N-(Benzyloxy carbonyl)-N′-(3- | Boc |
| tertbutoxycarbonyl amino | |
| propylene) thiourea | |
| N-(tert-Butoxy carbonyl)-N′-(6- | Fmoc |
| fluorenylmethoxycarbonyl amino | |
| hexyl) thiourea | |

### Formulae 1a, 1b and 1c: General Formula for Monomers for Obtaining Peptides, Oligomers and Oligomer-Peptide Hybrid Compounds

The present invention discloses peptides, oligomers and oligomer-peptide hybrid compounds also envisages within its scope monomers (Formulae 1a, 1b and 1c) for obtaining peptides, oligomers and oligomer-peptide hybrid compounds. Particularly, the Formula 1 is of Formulae 1a-1c as given below:

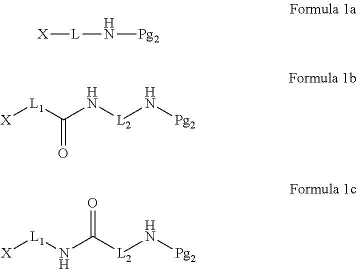

wherein X is

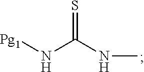

wherein linker groups “L, L1, L2,” is an aliphatic group containing propyl (C₃), butyl (C₄), hexyl (C₆), N-substituted alkyl amide, C₁-C₁₄₀ carbon atoms, selected from group comprising an alkyl group such as methylene, ethylene, propylene, C₄, C₅, C₆, C₇, C₈, C₉ or C₁₀; C₁-C₁₀, —C₂₀, —C₃₀, —C₄₀, —C₅₀-C₆₀, —C₇₀, —C₈₀, —C₉₀, —C₁₀₀, —C₁₁₀, —C₁₂₀, —C₁₃₀ or —C₁₄₀, alkyl; cycloaliphatic, heterocyclic, aromatic, aryl, alkylaryl, arylalkyl, oxyalkylene radicals; a polyalkylene radical optionally interrupted by one or more, preferably one, oxygen, nitrogen or sulphur atoms, functional groups, saturated or unsaturated cyclic moiety; or polyethers selected from the group comprising PEG/derivatives of PEG/analogues of PEG.
wherein linker groups comprise additional cationic moieties selected from the group comprising secondary or tertiary or quaternary amines, or guanidine, or aminoguanidine, diaminoguanidine or analogue, derivative cationic groups either in the backbone of the linker or side chain of the linker, the linker may additionally comprise an amide bond.
wherein protecting group Pg1 or Pg2 is independently selected from the group consisting of Pg1-Fmoc, Pg2-Boc, Pg1-Boc, Pg2-Fmoc, Pg1-Cbz.

Table 2 show compounds of Formulae 1, 1a, 1b and 1c and these are exemplary and illustrative compounds.

TABLE 2
Compounds of Formulae 1, 1a, 1b, 1c in the present invention

| Compound | | |
|---|---|---|
| no. | Structure of the compound | IUPAC name |
| 101 | 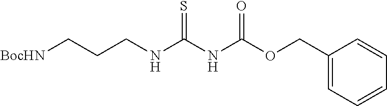 [SMILES: CC(C)(C)OC(=O)NCCCNC(=S)NC(=O)OCc1ccccc1] | N-(Benzyloxy carbonyl)-N′-(3- tertbutoxycarbonyl amino propylene) thiourea; |
| 102 | 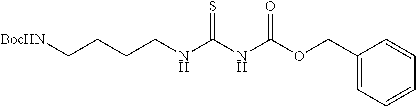 [SMILES: CC(C)(C)OC(=O)NCCCCNC(=S)NC(=O)OCc1ccccc1] | N-(Benzyloxy carbonyl)-N′-(4- tertbutoxycarbonyl amino butylene) thiourea; |
| 103 | 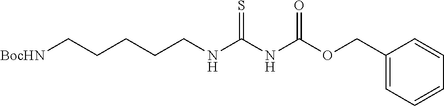 [SMILES: CC(C)(C)OC(=O)NCCCCCNC(=S)NC(=O)OCc1ccccc1] | N-(Benzyloxy carbonyl)-N′-(5- tertbutoxycarbonyl amino pentylene) thiourea; |
| 104 | 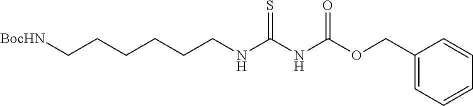 [SMILES: CC(C)(C)OC(=O)NCCCCCCNC(=S)NC(=O)OCc1ccccc1] | N-(Benzyloxy carbonyl)-N′-(6- tertbutoxycarbonyl aminohexylene) thiourea; |
| 105 | 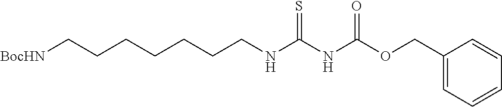 [SMILES: CC(C)(C)OC(=O)NCCCCCCCNC(=S)NC(=O)OCc1ccccc1] | N-(Benzyloxy carbonyl)-N′-(7- tertbutoxycarbonyl amino heptylene) thiourea; |
| 106 | 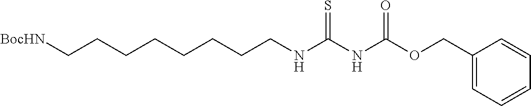 [SMILES: CC(C)(C)OC(=O)NCCCCCCCCNC(=S)NC(=O)OCc1ccccc1] | N-(Benzyloxy carbonyl)-N′-(8- tertbutoxycarbonyl amino octylene) thiourea; |
| 107 | 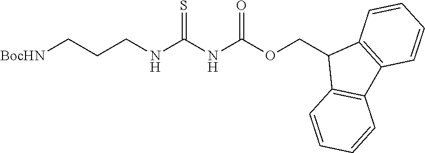 [SMILES: CC(C)(C)OC(=O)NCCCNC(=S)NC(=O)OCC1c2ccccc2-c2ccccc21] | N-(-Fluorenyl methoxy carbonyl)- N′-(3-tertbutoxycarbonyl amino propylene) thiourea; |
| 108 | 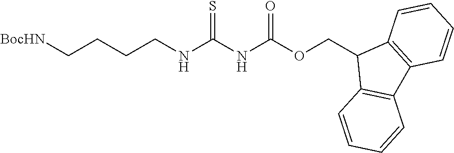 [SMILES: CC(C)(C)OC(=O)NCCCCNC(=S)NC(=O)OCC1c2ccccc2-c2ccccc21] | N-(9-Fluorenyl methoxy carbonyl)- N′-(4-tertbutoxycarbonyl amino butylene) thiourea; |
| 109 | 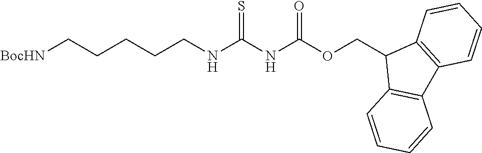 [SMILES: CC(C)(C)OC(=O)NCCCCCNC(=S)NC(=O)OCC1c2ccccc2-c2ccccc21] | N-(9-Fluorenyl methoxy carbonyl)- N′-(5-tertbutoxycarbonyl amino pentylene) thiourea; |
| 110 | 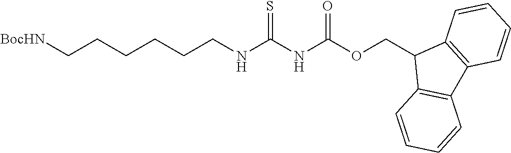 [SMILES: CC(C)(C)OC(=O)NCCCCCCNC(=S)NC(=O)OCC1c2ccccc2-c2ccccc21] | N-(9-Fluorenyl methoxy carbonyl)- N′-(6-tertbutoxycarbonyl aminohexylene) thiourea; |
| 111 | 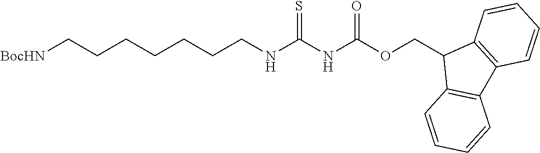 [SMILES: CC(C)(C)OC(=O)NCCCCCCCNC(=S)NC(=O)OCC1c2ccccc2-c2ccccc21] | N-(9-Fluorenyl methoxy carbonyl)- N′-(7-tertbutoxycarbonyl amino heptylene) thiourea; |
| 112 | 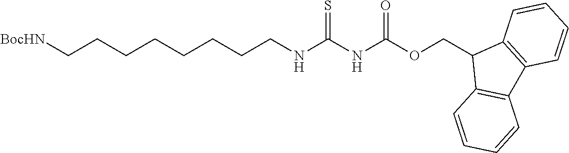 [SMILES: CC(C)(C)OC(=O)NCCCCCCCCNC(=S)NC(=O)OCC1c2ccccc2-c2ccccc21] | N-(9-Fluorenyl methoxy carbonyl)- N′-(8-tertbutoxycarbonyl amino octylene) thiourea; |
| 113 | 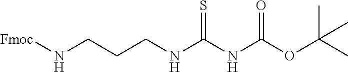 | N-(tertbutoxy carbonyl)-N′-(3- fluorenylmethoxycarbonyl amino propyl) thiourea; |
| 114 | 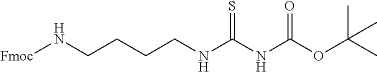 | N-(tertbutoxy carbonyl)-N′-(4- fluorenylmethoxycarbonyl amino butyl) thiourea; |
| 115 | 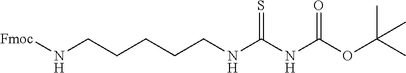 | N-(tertbutoxy carbonyl)-N′-(5- fluorenylmethoxycarbonyl amino pentyl) thiourea; |
| 116 | 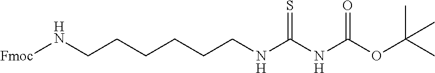 | N-(tertbutoxy carbonyl)-N′-(6- fluorenylmethoxycarbonyl amino hexyl) thiourea; |
| 117 | 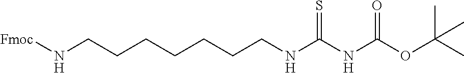 | N-(tertbutoxy carbonyl)-N′-(7- fluorenylmethoxycarbonyl amino heptyl) thiourea; |
| 118 | 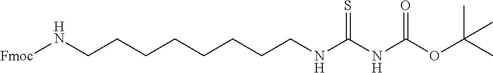 | N-(tertbutoxy carbonyl)-N′-(8- fluorenylmethoxycarbonyl amino octyl) thiourea; |
| 119 | 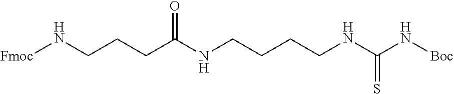 | 4-fluorenylmethoxycarbonyl amino- N-(4-(3-tertbutyloxycarbonyl thioureido)butyl) butanamide; |
| 120 | 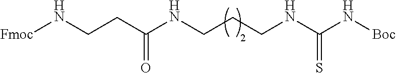 | 3-fluorenylmethoxycarbonyl amino- N-(4-(3-tertbutyloxycarbonyl thioureido)butyl) propanamide; |
| 121 | 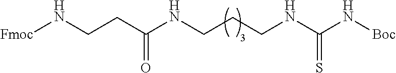 | 3-fluorenylmethoxycarbonyl amino- N-(5-(3-tertbutyloxycarbonyl thioureido)pentyl) propanamide; |
| 122 | 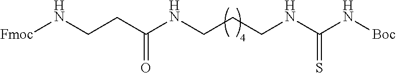 | 3-fluorenylmethoxycarbonyl amino- N-(6-(3-tertbutyloxycarbonyl thioureido)hexyl) propanamide; |
| 123 | 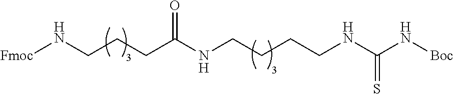 | 6-fluorenylmethoxycarbonyl amino- N-(6-(3-tertbutyloxycarbonyl thioureido)hexyl) hexanamide; |
| 124 | 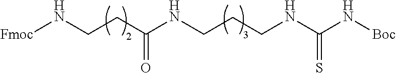 | 4-fluorenylmethoxycarbonyl amino- N-(5-(3-tertbutyloxycarbonyl thioureido)pentyl) butanamide; |
| 125 | 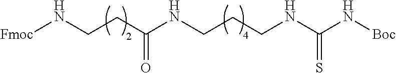 | 4-fluorenylmethoxycarbonyl amino- N-(6-(3-tertbutyloxycarbonyl thioureido)hexyl) butanamide. |
| 126 | 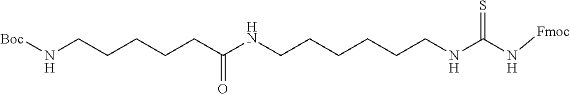 | 6-tertbutyloxycarbonyl amino-N-(6- (3-fluorenylmethoxycarbonyl thioureido)hexyl) hexanamide |
| 127 | 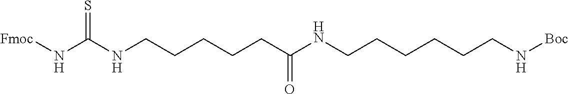 | 6-(3-fluorenylmethoxycarbonyl thioureido)-N-(6- tertbutyloxycarbonylamino)hexyl) hexanamide |

Formula 2: General Formula of Cationic Guanidine Based Peptide, Oligomer, and Oligomer-Peptide Hybrid Compound, Obtained from Monomer of Formulae 1a, 1b, 1c

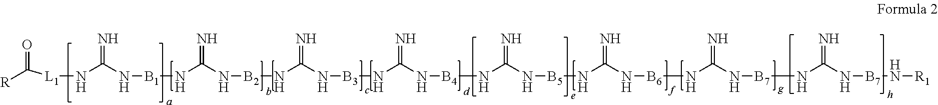

wherein “B1-B7” is independently selected from the L or

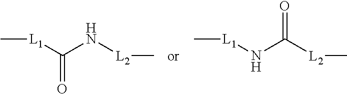

or C2-C8
wherein B1-B7 is L,
or

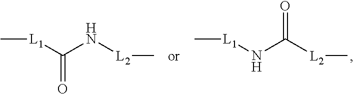

or combination of L and

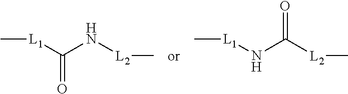

where R is independently selected from the group consisting of NH₂, OH;
where linker groups L₁, L₂, L₃ are independently selected from the group consisting of C2-C8,

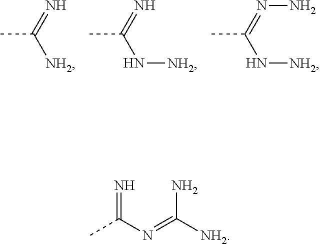

where-R1 is independently selected from —H, -acetyl, where R1 is defined such a way that it yields guanidine, aminoguanidine, biguanidine, diaminoguanidine, Additionally, R1 is added by linkage to aliphatic, cycloaliphatic heterocyclic, heterocyclic, aryl, alkylaryl, arylalkyl, oxyalkylene radicals. In addition, end groups of macromolecules may be modified by linkage to receptor ligands, dextrans, cyclodextrins, fatty acids or fatty acid derivatives, lipids, cholesterol or cholesterol derivatives or polyethylene glycol (PEG), fluorophore (like FITC, Alexa fluor —NHS ester).
a, b, c, d, e, f, g, h each is independently selected from any integer chosen from 0-100, for example, from 0, 1, 2, 3, 4 or 5 to 10, 20, 30, 40, 50, 60, 70, 80, 90, 100, where the sum of a-h is at least 1.

Compounds of Formula 2 include but are not limited to the compounds shown below in Tables 3-5.

TABLE 3
Example of compounds of Formula 2

| Compound | |
|---|---|
| no. No. | Compound |
| 2001 | 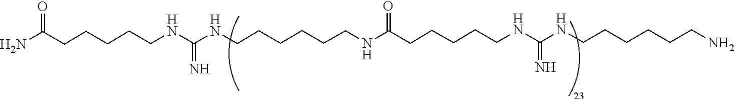 |
| 2002 | 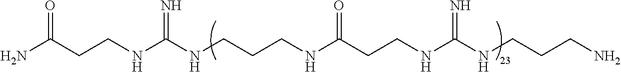 |
| 2003 | 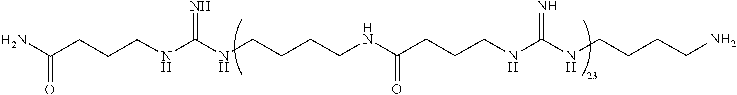 |
| 2004 | 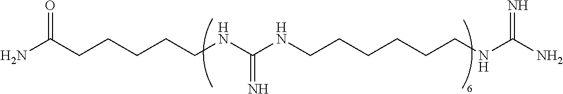 |
| 2005 | 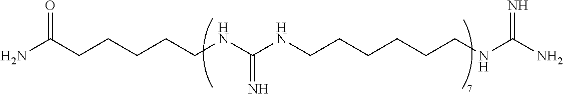 |
| 2006 | 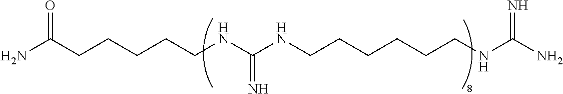 |
| 2007 | 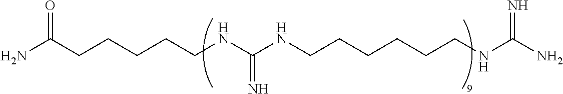 |
| 2008 | 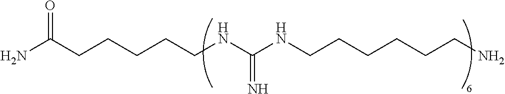 |
| 2009 | 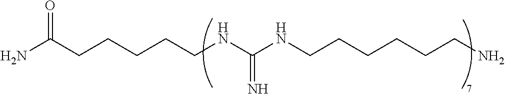 |
| 2010 | 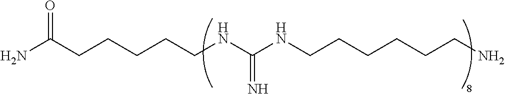 |
| 2011 | 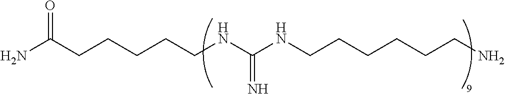 |
| 2012 | 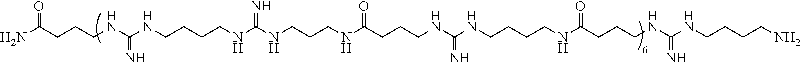 |

TABLE 4
Example of oligoguanidine compounds of Formula 2

| Oligoguanidine | L1 | L2 | L3 | n |
|---|---|---|---|---|
| 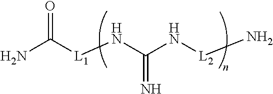 | —(CH₂)₅ | —(CH₂)₆ | | 2-30 |
| 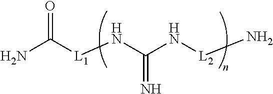 | —(CH₂)₃ | —(CH₂)₄ | | 2-30 |
| 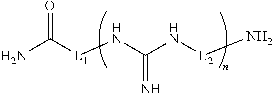 | —(CH₂)₂ | —(CH₂)₃ | | 2-30 |
| 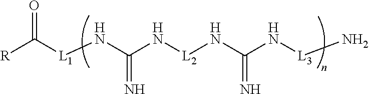 | —(CH₂)₅ | —(CH₂)₆ | (CH₂)₃ | 1-30 |
| 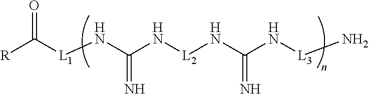 | —(CH₂)₅ | —(CH₂)₆ | (CH₂)₄ | 1-30 |
| 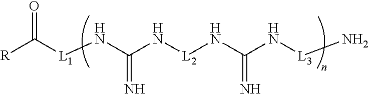 | —(CH₂)₅ | —(CH₂)₃ | (CH₂)₄ | 1-30 |

TABLE 5
Example of guanidine backbone peptides of Formula 2

| Guanidine backbone peptides | L1 | L2 | L3 | n |
|---|---|---|---|---|
| 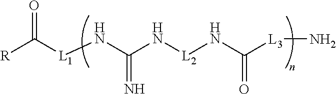 | —(CH₂)₅ | —(CH₂)₆ | (CH₂)₅ | 2-30 |
| 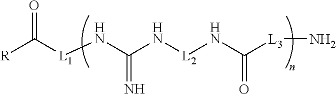 | —(CH₂)₃ | —(CH₂)₄ | (CH₂)₃ | 2-30 |
| 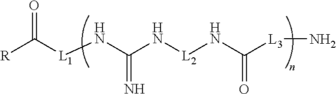 | —(CH₂)₂ | —(CH₂)₃ | (CH₂)₂ | 2-30 |
| 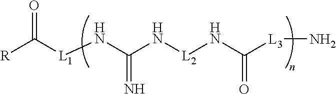 | —(CH₂)₅ | —(CH₂)₆ | (CH₂)₂ | 2-30 |
| 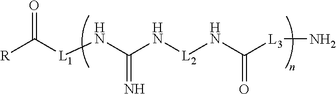 | —(CH₂)₅ | —(CH₂)₆ | (CH₂)₃ | 2-30 |
| 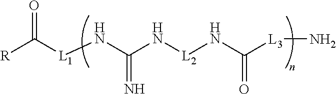 | —(CH₂)₅ | —(CH₂)₄ | (CH₂)₂ | 2-30 |

Formula 3: General Formula of Cationic Guanidine Based Oligomer-Peptide Hybrid Compound, Obtained from Monomer of Formulae 1a, 1b and 1c

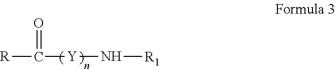

wherein Y is selected from
(AA1)ₘ₁-(AA2)ₘ₂-(AA3)ₘ₃-(AA4)ₘ₄-(AA5)ₘ₅-(AA6)ₘ₆-(OG1)ₙ₁-(OG2)ₙ₂-(OG3)ₙ₃-(OG4)ₙ₄-(OG5)ₙ₅-(OG6)ₙ₆-
(OG1)ₙ₁-(OG2)ₘ₂-(OG3)ₘ₃-(OG4)ₘ₄-(OG5)ₘ₅-(OG6)ₙ₆-(AA1)ₘ₁-(AA2)ₘ₂-(AA3)ₘ₃-(AA4)ₘ₄-(AA5)ₘ₅-(AA6)ₘ₆
(AA1)ₘ₁-(OG1)ₙ₁-(AA2)ₘ₂-(OG2) 12-(AA3)ₘ₃-(OG3)ₙ₃-(AA4)ₘ₄-(OG4)ₙ₄-(AA5)ₘ₅-(OG5)ₙ₅-(AA6)ₘ₆-(OG6)ₙ₆
(AA1)ₘ₁-(OG)ₙ₁; -(OG1)ₘ₁-(AA1)ₙ₁-; -(OG1)ₘ₁-(AA1)ₙ₁-(OG2)ₘ₂-; or -(AA1)ₘ₁-(OG1)ₙ₁-(AA2)ₘ₂-;
wherein
m1, m2, m3, m4, m4, m6, n1, n2, n3, n4, n5, n6 are integers independently selected from 0, 1, 2 . . . 100 and n is an integer independently selected from 1 to 100; m1, n1, m2 and n2 is similar or different; where the sum of m1-m6 and n1-n6 is at least 2;
R together with the carbonyl group to which it is attached is independently selected from —H, free acid, amine, amide, acid salt, ester or functionalized carbonyl group;
R1 is independently selected from —H, -acetyl,

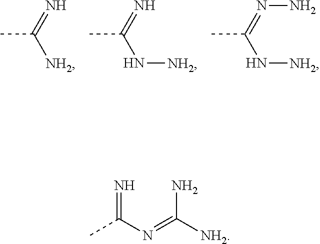

R1 is designed such a way that it yields guanidine, aminoguanidine, biguanidine, diaminoguanidine, Additionally, R1 is added by linkage to aliphatic, cycloaliphatic heterocyclic, heterocyclic, aryl, alkylaryl, arylalkyl, oxyalkylene radicals. In addition, end groups may be modified by linkage to receptor ligands, dextrans, cyclodextrins, fatty acids or fatty acid derivatives, lipids, cholesterol or cholesterol derivatives or polyethylene glycol (PEG), fluorophore (like FITC, Alexa fluor —NHS ester) and
OG1, OG2, OG3, OG4, OG5, OG6 is independently selected from oligomer residue or guanidine backbone amino acid residue arising from the monomer of Formula 1a or Formula 1b and 1c, respectively;
AA1, AA2, AA3, AA4, AA5, AA6 is amino acids residue independently selected from any of the natural amino acid residues (D and L form)/synthetic amino acid, such that the AA1 (amino acid residue) is present at any position in the oligomer, for example, at C-terminus, N-terminus, in between guanidine group of the oligomer residue.

The oligomers and peptides in Formula 2 and Formula 3 is in the form of a linear chain, branched chain, dendrimer forms and is in the form of a cyclic structure.

Compounds of Formula 3 include but are not limited to those shown in Tables 6 and 7

TABLE 6
Example of compounds of Formula 3

| Compound | |
|---|---|
| No. | Compound |
| 3001 | 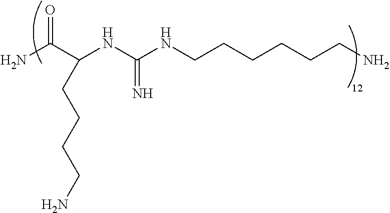 |
| 3002 | 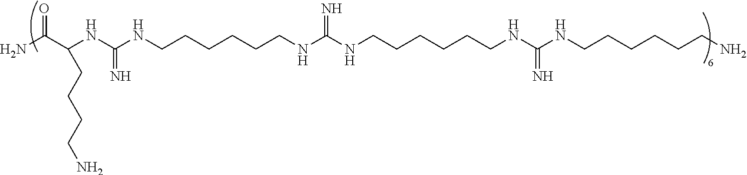 |
| 3003 | 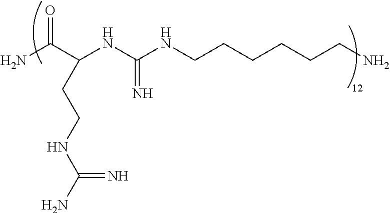 |
| 3004 | 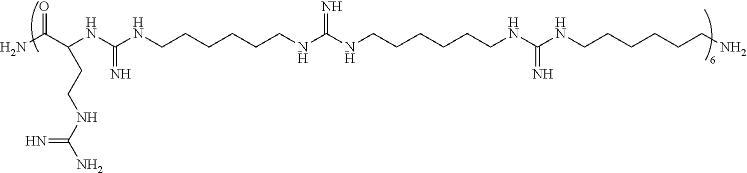 |
| 3005 | 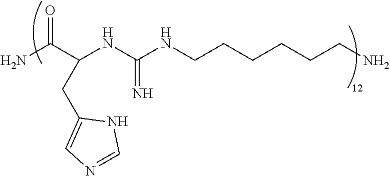 |
| 3006 | 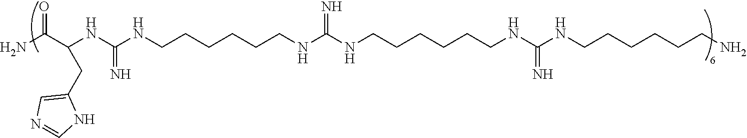 |

TABLE 7
Peptide-oligomer hybrid compounds of Formula 3

| Peptide-oligomer hybrids | L1 | L2 | L3 | L4 | m | n | x |
|---|---|---|---|---|---|---|---|
| 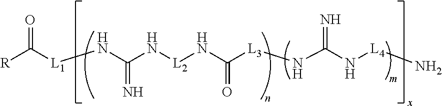 | —(CH₂)₅ | —(CH₂)₆ | —(CH₂)₅ | —(CH₂)₆ | 2-30 | 2-30 | 2-30 |
| 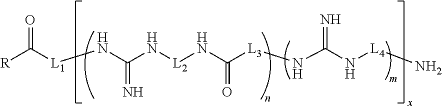 | —(CH₂)₃ | —(CH₂)₄ | —(CH₂)₃ | —(CH₂)₄ | 2-30 | 2-30 | 2-30 |
| 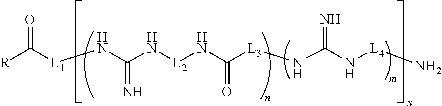 | —(CH₂)₂ | —(CH₂)₃ | —(CH₂)₂ | —(CH₂)₃ | 2-30 | 2-30 | 2-30 |
| 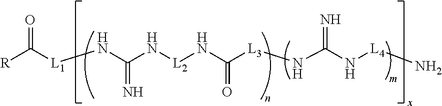 | —(CH₂)₅ | —(CH₂)₆ | —(CH₂)₂ | —(CH₂)₆ | 2-30 | 2-30 | 2-30 |
| 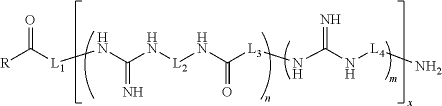 | —(CH₂)₅ | —(CH₂)₆ | —(CH₂)₃ | —(CH₂)₆ | 2-30 | 2-30 | 2-30 |
| 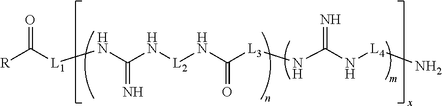 | —(CH₂)₃ | —(CH₂)₄ | —(CH₂)₂ | —(CH₂)₄ | 2-30 | 2-30 | 2-30 |
| 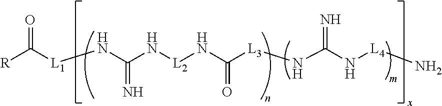 | —(CH₂)₂ | —(CH₂)₃ | —(CH₂)₃ | —(CH₂)₃ | 2-30 | 2-30 | 2-30 |

### B. PROCESS FOR SYNTHESIS OF THE COMPOUNDS OF THE PRESENT Invention

### Process for Synthesis of the Monomers

In an embodiment the present invention discloses a process for preparing the monomers and the oligomers, peptides and peptide-oligomers with cationic group/s in the backbone.

The monomers of the present invention may be used to make oligomers and peptides either by solid phase or solution phase. Such compounds will have net positive charge on the oligomer or peptide and confer several novel properties and advantages over existing strategies. The cationic peptide backbone described here should not be confused with positive charge coming from the side chain of lysine or arginine or analogues. The monomers of the present invention are compatible with standard solid (as shown in the below example) or solution phase synthesis. The process comprises the steps of:
i. treating a diamine compound (10 equiv.) with di-tertbutyl carbonate orthogonal amine protecting group (1 equiv.) in chloroform a solvent (0-10° C., for 12-24 hours) to provide intermediate (A) wherein one of the diamine is selectively protected; and
ii. treating the intermediate A (1 equiv.) obtained in step (i) with protected isothiocyanate (1.1 equiv.), (B) in tetrahydrofuran (THF) solvent (4-25° C., for 12-24 hours) to obtain the compound of Formula 1a

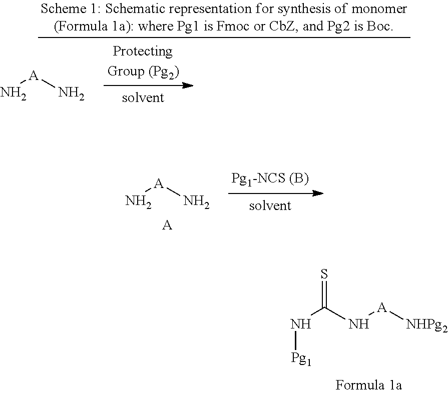

The process for preparing compounds of Formula 1a, said The process may comprise the steps of:
i. To a precooled (0-10° C.) solution of 1,6-diamino hexane (10 equiv.) in 100 volumes of non-polar solvent (chloroform), di-tertbutyl carbonate (1 equiv.) in chloroform (10 volume) was added dropwise, and stirred for 12-24 hours. After completion of reaction the intermediate (A) was isolated in 60-80% yield wherein one of the diamines is selectively protected; and
ii. To the solution of intermediate A in non-polar solvent (tetrahydrofuran, 10 volume) obtained in step 1, 1 equiv. of fluorenylmethoxycarbonyl isothiocyanate (Fmoc-NCS was added and stirred for 0.5-3 hours at 4-25° C. After completion of reaction as monitored over TLC. Hexane (non-polar solvent 200 volume) was added and stirred to obtain the precipitated compound of Formula 1 in 50-70% yield. In a similar manner, benzyloxycarbonyl isothiocyanate (Cbz-NCS) has also been used to synthesize another form of Formula 1.

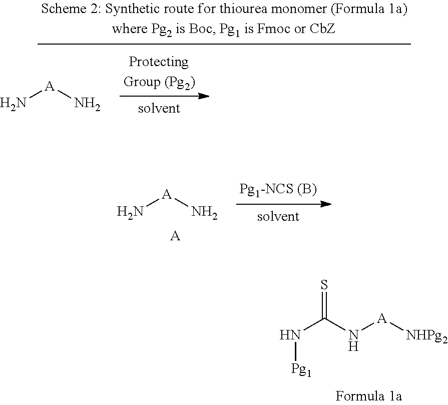

A process for preparing the compounds of Formula 1a comprises the steps of:
i. The tert-butylcarbamate (Boc-amine, 1 equiv.) was treated with carbon disulfide (5-10 equiv, CS₂) in presence of KOH (1 equiv.) using hexane:ethylacetate (2:1) solvent mixture at 10-25° C. for 12-24 hours to form potassium tert-butylcarbamodithioate. Then, tert-butylcarbamodithioate was treated with sodium persulphate (1 equiv.) in aqueous media with ice cold condition for 10-30 min and completion of reaction was monitored with TLC. The tert-butyloxycarbonyl isothiocyanate (Boc-NCS) was extracted with dichloromethane and used in next step reaction without isolation.
ii. Second method to synthesize Boc-NCS from tert-butylcarbamodithioate in organic solvent was also employed. In brief, tert-butyl carbamodithioate (1 equiv.) was suspended in THF (10 volume) and 0.9 equiv. of Boc-anhydride was added in ice cold condition and reaction mixture was stirred for 15 min. Then, solution of (0.1-0.3 equiv.) 4-dimethylamino pyridine (DMAP) in THF was added and left for stirring for another 15 mins. The reaction mixture was filtered and used for the next step immediately.
iii. The solution of 1,6-diamino hexane (3 equiv.) in DCM was treated Boc-NCS (dropwise addition) at 4-25° C. to yield in 30-50% of 1-(6-aminohexyl)-3-(tert-butyloxycarbonyl) thiourea (A). Then, further free amine of thiourea (A) was reacted with Fmoc-OSu (1.1 equiv.) and after completion of reaction as monitored over TLC, the solvent was evaporated and reconstituted in nonpolar solvent (DCM) and organic phase was treated with 10% citric acid followed by distilled water and brine solution. The organic phase was dried over sodium sulphate and compound was isolated after evaporating the organic phase. The compound was purified over flash chromatography 30% ethylacetate: Hexane (30-50% yield). The synthesize monomer (Formula 1a) having Fmoc as Pg1 and Boc as Pg₂.

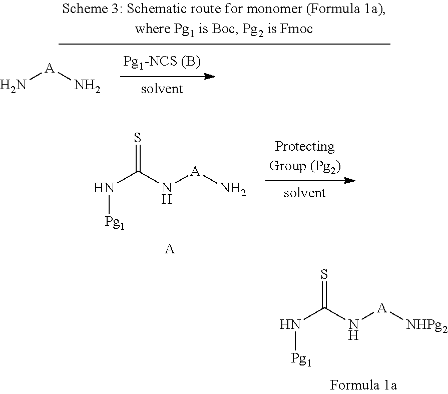

For synthesis of compounds with Formula 1b with amide bond, N-Fmoc-6-amino hexanoic acid (1 equiv.) in nonpolar solvent (DCM, 10 volume) was treated with 1.1 equiv. of 1-Ethyl-3-[3-dimethylaminopropyl] carbodiimide hydrochloride (EDC or EDAC) and 1.1 equiv. of HOBt for 1-3 hours at 10-30° C. The resulting activated carboxylic acid group was reacted with N-boc-1,6 diamine at 20-30° C. for 12-24 hours. After completion of reaction as monitored over TLC, the organic phase was washed with 10% citric acid followed by 10% sodium bicarbonate, distilled water and brine solution. The organic phase was dried over sodium sulphate and evaporated to obtain the compound (A) in 50-70% yield. Further compound was treated with 20% piperidine in DMF for 1-4 h to deprotect the fmoc group. After completion of the reaction, the compound was precipitated by adding water and filtered, dried. The solution of deprotected compound in non-polar solvent (THF), Fmoc-NCS (1 equiv.) was added and stirred for 0.5-2 hours at 20-30° C. The compound was precipitated by addition of hexane (200 volume) and filtered to yield compound with Formula 1b in 30-50% yield.

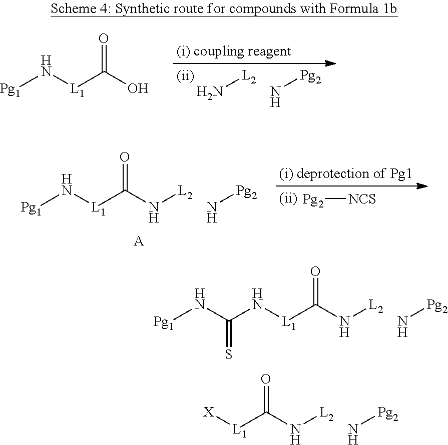

For synthesis of compounds with Formula 1c, the compound A from Scheme 4 was treated with DCM;TFA (1:1) to deprotect Pg2 group (Boc) and precipitated in diethylether, the obtained compound was reacted with Boc-NCS in presence of organic base (such as trimethylamine, diisopropyl ethyl amine) to yield the compound with Formula 1c. Alternatively, N-Boc-6-amino hexanoic acid (1 equiv.) in nonpolar solvent (DCM, 10 volume) was treated with 1.1 equiv. of 1-Ethyl-3-[3-dimethylaminopropyl] carbodiimide hydrochloride (EDC or EDAC) and 1.1 equiv. of HOBt for 1-3 h at 10-30° C. The resulting activated carboxylic acid group was reacted with 1,6 diamine (3 equiv.) at 20-30° C. for 12-24 h. After completion of reaction as monitored over TLC, the organic phase was washed with distilled water and brine solution. The organic phase was dried over sodium sulphate and evaporated to obtain the compound (A) in 50-70% yield. The compound C (lequiv. in THF) was treated with Fmoc-NCS (1 equiv.) for 0.5-2 h at 20-30° C. After completion of the reaction, the final compound with Formula 1c was precipitated by addition of hexane (200 volume) and filtered, dried in 30-50% yield.

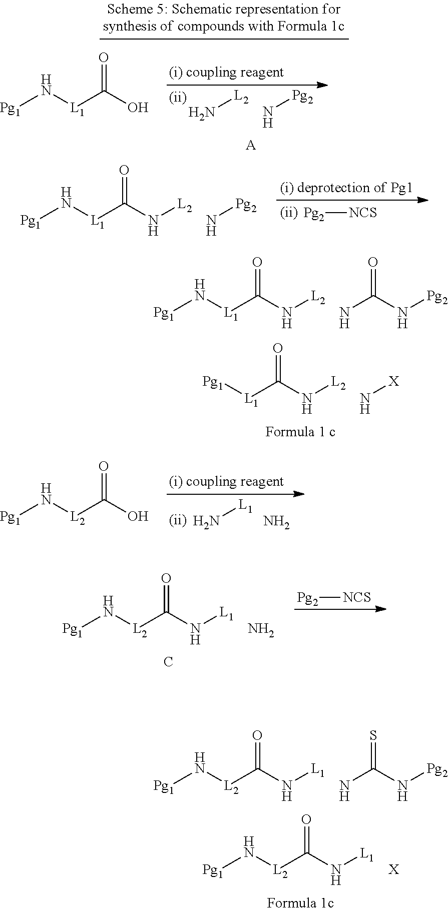

### Processes for Synthesis of Cationic Guanidine Based Peptide, Oligomer, Oligomer-Peptide Hybrid Compounds

Addition of these cationic backbone polymers or peptides is done during synthesis. For example, when a desired length of compounds are synthesized and bound to resin, then the peptidomimetic analogues is added by step-by-step addition of these agents using monomers of Formulae 1a, 1b and 1c as shown in below example:

The oligomer of Formula 2 is synthesized in a controlled manner as shown in Scheme 6, such that the obtained oligomer is pure and free from side products suitable for pharmaceutical applications.

### Formula 2

Formula 2 can give both peptides or oligomers depending on the monomer used (Formula 1a or 1b or 1c)

The process includes the steps of:
1. The process includes the steps of:
   i. placing MBHA (Methylbenzhydrylamine) resin in a peptide synthesizer vessel; Wang resin, Rink amide MBHA resins can also be used
   ii. swelling the resin with suitable solvent (DCM) followed by washing with solvent(s);
   iii. carboxylic acid coupling method (coupling method A) wherein the resin is treated with acid reactant (C) having Fmoc as protected group using HBTU (coupling agent) and DIPEA (a base) in DMF (solvent) to provide intermediate (D); other protecting groups, for example, Boc are used.
   iv. capping of free amines on resin-optional capping of free amino groups using acetic anhydride in DMF (a solvent);
   v. deprotection method wherein deprotection of intermediate (D) using 20% piperidine (for Fmoc)/50% TFA-5% m-cresol-45% DCM (for Boc) followed by washing with DMF, DCM or their mixture and last washing with DCM
   vi. coupling for monomer (Formula 1) (coupling method B) wherein the monomer (Formula 1) was added to the mixture of 12/TMP (1:3 equiv.) or N-iodosuccinimide (NIS) or 1,3-Diiodo-5,5-Dimethylhydantoin (DIH) or any other desulphurizing agent in a solvent medium and the free amine containing resin is treated to form the coupled product (E);
   vii. deprotection of coupled product (E) followed by washing and then reaction with second unit of monomer of Formula 1 as claimed in claim 1
   viii. repetition of step F until the oligomer with desired number of repeating units is formed; and
   ix. cleaving of the resin followed by precipitation and desalted using C₁₈ silica and lyophilized using temperature (−20° C. to −110° C.) and pressure (range of 0.01 to 1.0 mbar) to obtain the compound of Formula 2 as claimed in claim 4.

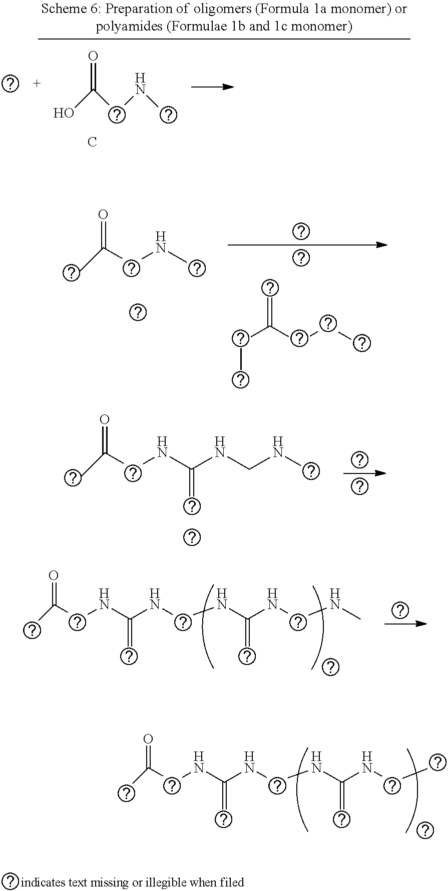

### Processes for Synthesis of Cationic Guanidine Based Oligomer-Peptide Hybrid Compounds

To obtain the oligomer-peptide hybrid compound of Formula 3 in a controlled way, the solid phase synthesis is preferable using thiourea monomers of Formula 1 and natural/unnatural amino acid monomers:

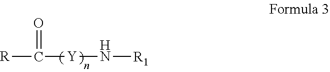

The process of synthesis of oligomer-peptide hybrid using Formula 1 and amino acid (synthetic/natural) involves the following steps:
i. treating MBHA resin (A) with acid reactant (B) in the presence of coupling agent and a base in a solvent to the corresponding amide followed by deprotection;
ii. reacting intermediate from step i with monomer of Formula 1 using TMP/I₂ followed by deprotection to give guanidine-based amine intermediate C;
iii. acid-amine coupling of guanidine-based intermediate C with amine-protected amino-acid, followed by deprotection to obtain coupled product D;
iv. reacting coupled product D with second unit of monomer of Formula 1 using TMP/I₂, deprotection and then again reaction with 3rd unit of monomer of Formula 1 using TMP/I₂ and deprotection;
v. continuation of reaction until introduction of nth unit of monomer of Formula 1 and final deprotection and resin cleavage.
vi. The sequence of coupling with amino-acid or monomer and the number of amino-acid residue and monomer unit can vary according to the targeted structure of the oligomer-peptide hybrid compound.

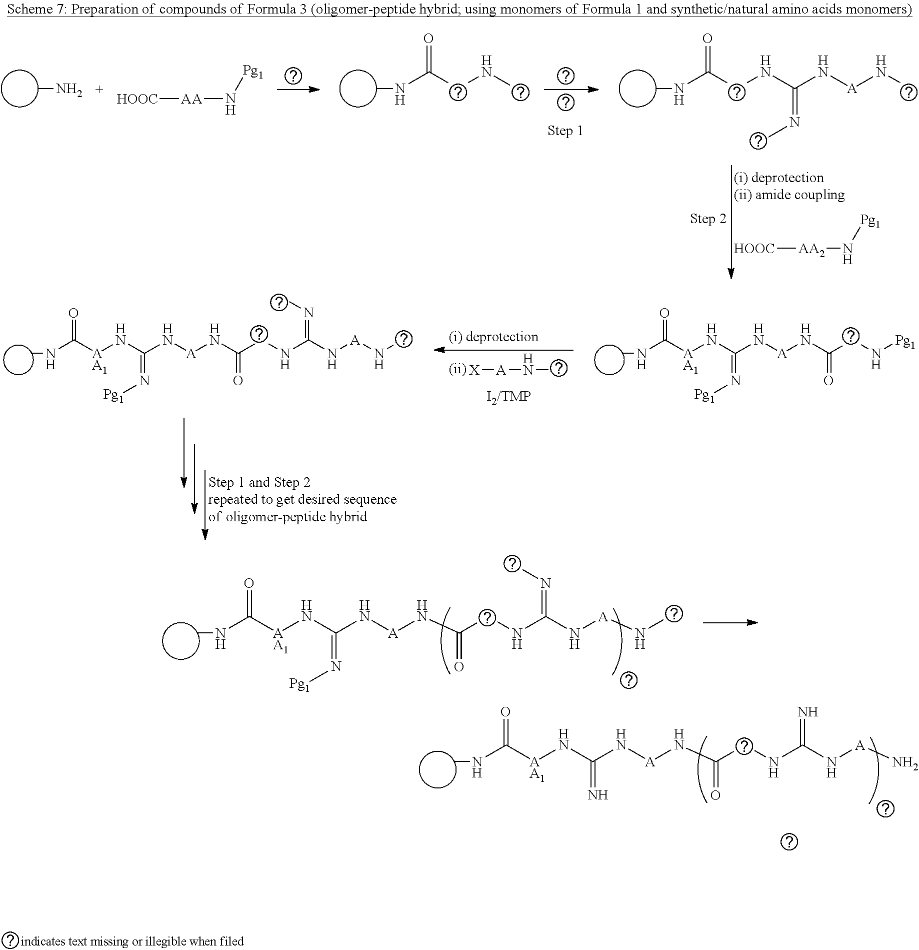

Using monomers of Formula 1 and lysine amino acid monomer, the oligomer-peptide shown in Scheme 8 is synthesized.

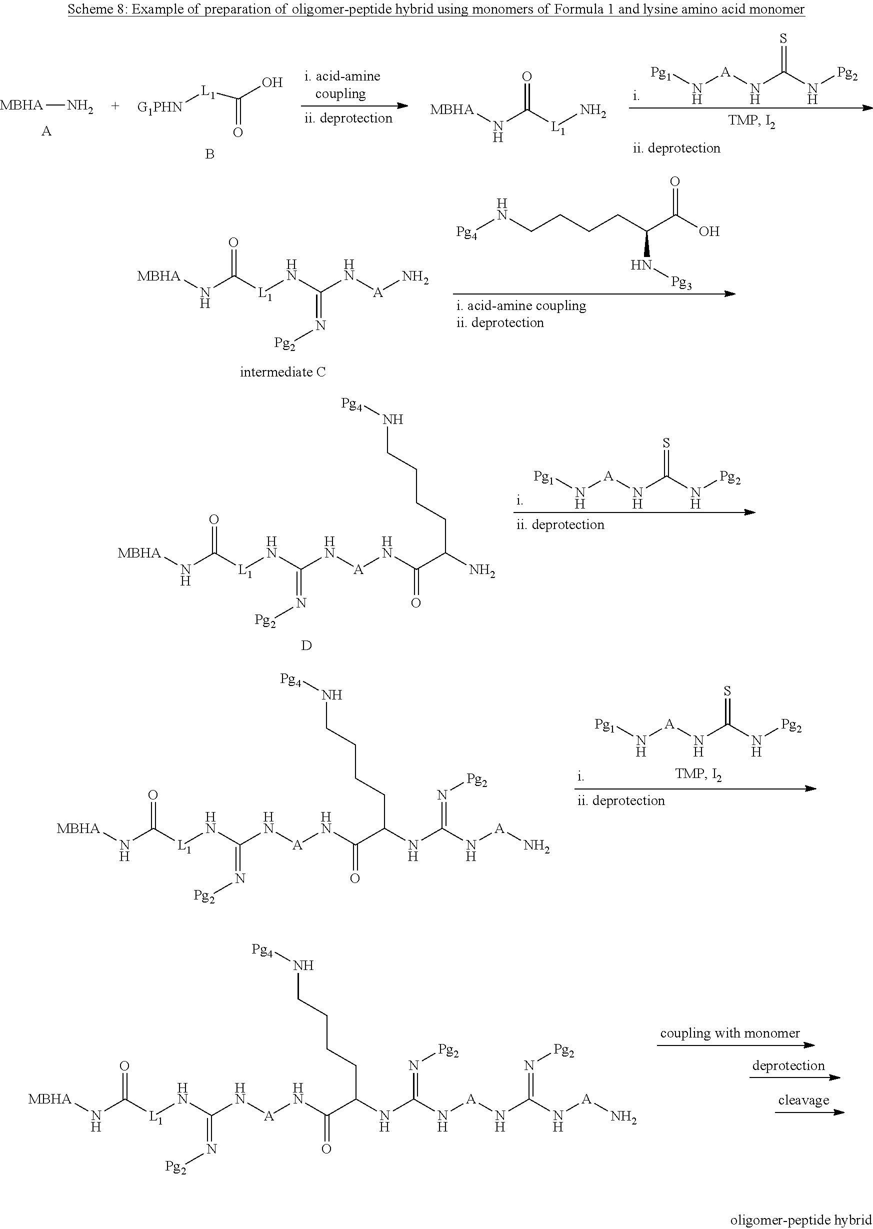

The present invention also described another method to produce cationic backbone peptides using monomers of Formulae 1b or 1c. The process involves the following steps:
i. treating MBHA resin with acid reactant (C) in the presence of coupling agent and a base in a solvent to the corresponding amide followed by deprotection;
ii. reacting intermediate D after deprotection with monomer of Formula 1b or 1c using TMP/I₂ to give guanidine-based amine intermediate E;
iii. the deprotection of Pg1 followed by coupling of formula 1b or 1c using TMP/I₂, the same process repeated until desired length of the compound being made. Then final cleavage of the compound, precipitation and followed by desalting to yield purified compound.

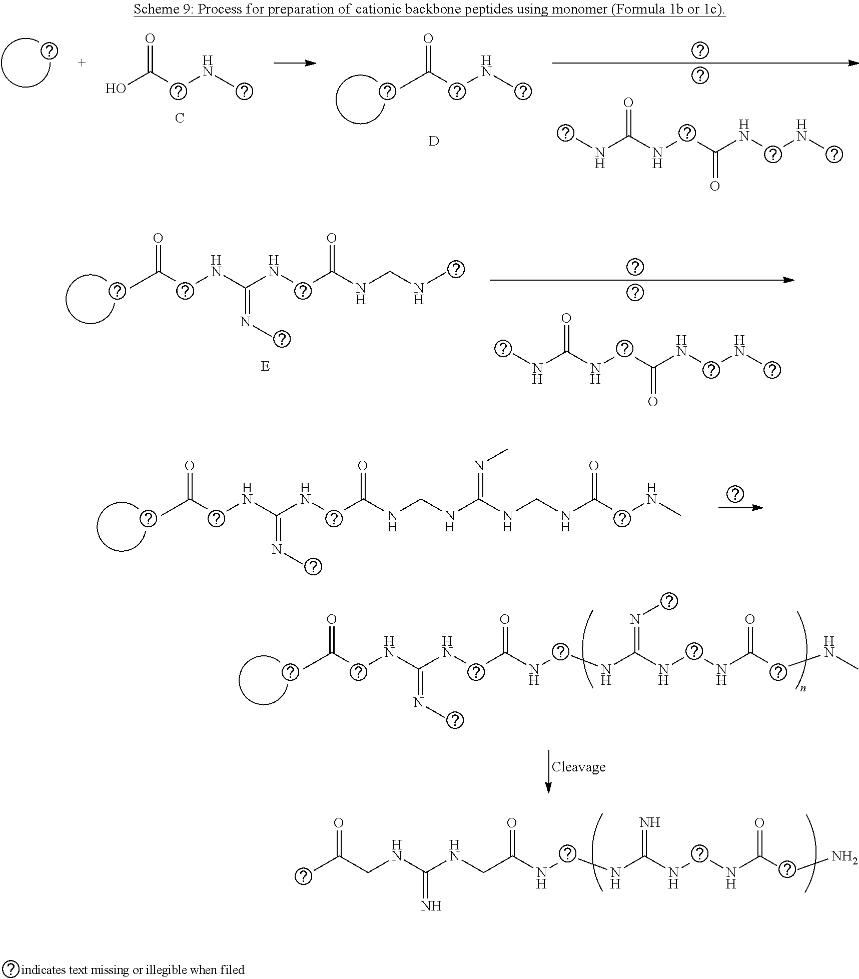

The present invention also described another method to produce cationic backbone peptides using monomers of Formula 1a. The process involves the following steps:
i. treating MBHA resin with acid reactant (C) in the presence of coupling agent and a base in a solvent to the corresponding amide followed by deprotection;
ii. reacting intermediate D after deprotection with monomer of Formula 1a using TMP/I₂ to give guanidine-based amine intermediate E;
iii. the intermediate E after deprotection reacted with amine protected unnatural amino acid (F) in presence of HBTU (coupling reagent) and DIPEA (a base)
iv. then again, deprotection followed by coupling of G using TMP/I₂ to give guanidine.
v. The amide and thiourea couplings were proceeded to yield guanidine-polyamide using Formula 1a.

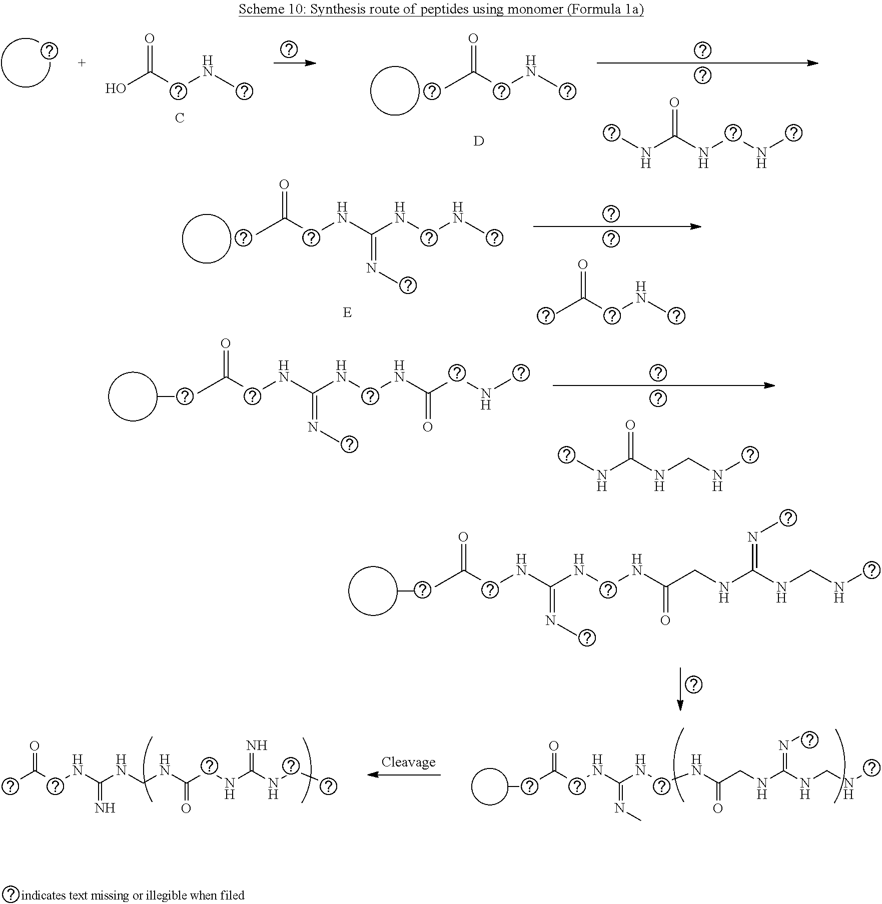

The resulting compounds are homo peptides or oligomers when the same type of monomer is used. Alternatively, the resulting compounds is heteropeptides or oligomers when more than one type of monomer is used.

### Modifications in the End Groups of Peptides

Peptides can have N-terminal and C-terminal ends, with various modifications.

N-terminus end (often referred to as an amine end): Usually amino terminus of the peptide has a free amine (NH₂ group). The amine end group is modified to secondary/tertiary/quaternary amine, acetyl, amide, amidine, guanidine, aminoguanidine, diaminoguanidine, diguanidine, thiourea, urea, and derivatives/analogues (including methylguanidine, dimethylguanidine, trimethylguanidine, tetramethylguanidine, cyanoguanidine and their analogues/derivatives). In some instance, amine terminus is modified with a suitable moiety using any groups described for L1 or L2. In some instances, end groups in the peptide may be modified by linkage to receptor ligands (e.g., RGD) motif, folic acids, etc.) or receptor agonists or another synthetic peptide made from natural amino acids/analogues. These may be single amino acid or up to 50 amino acids. The end group is conjugated to carbohydrates (such as, chitosan/its derivatives, dextrans/derivatives, hyaluronic acids, alginates, cyclodextrins, chondroitin, heparin, heparan, etc.), lipids, fatty acids or fatty acid derivatives, cholesterol or cholesterol derivatives or polyethylene glycol (PEG). In some instance, the peptide is conjugated to nucleic acids or analogues, synthetic antisense/antigene nucleic acids like PNA, LNA, or small molecule drugs, imaging agents, contrast agents and diagnostic molecules. Sometimes, new end groups in the form of peptides is arginyl-glycyl-aspartic acid (be joined by post-synthetic coupling of more than one peptide.

C-terminus (carboxyl end) of the peptide is a free acid or amide or ester, additional end groups are possible depending up on the solid support (resin) used in the peptide synthesis. Also, the C-terminus of the peptide is modified to have above-listed modifications (those described for N-terminus).

Salts: The peptides have associated salts. Examples are limited to trifluoroacetic anhydride (TFA), halides (chloride, fluoride, iodide, bromide), phosphates, sulphates, oxalates, carboxylates and fatty acids. The peptides is in the form of a linear chain, branched chain, multiple antigenic format (MAP) or dendrimeric forms, and in some instance, peptide is cyclic.

The peptides/hybrid peptides/peptide-oligomers of the invention is either linear or branched/dendrimeric forms, broadly classified as follows.
a. Linear/branched/dendrimeric homogenous cationic backbone peptides (made from one type of cationic backbone monomers are described).
b. Linear/branched/dendrimeric heterogeneous cationic backbone peptides (made from more than one cationic backbone monomers are described).
c. Linear/branched/dendrimeric heterogeneous hybrid peptides (made from one or more than one type of cationic backbone monomers are described here, and one or more than one natural amino acid monomers/derivatives or a suitable linker).
d. Linear/branched/dendrimeric hybrid peptide-oligomers (made from a combination of one or more than one type of cationic backbone monomers, natural amino acid monomers and their derivatives, oligomer monomers with or without a suitable linker).

Such compounds possess variety of biological functions, and these are synthesized using standard solid phase or solution phase peptide.

The oligomers/peptides/oligomer-peptide obtained through this scheme are compatible with solid and solution phase peptide synthesis. The present invention also describes the use of these monomers to make novel peptides with cationic moieties in the backbone (main chain). Such peptides are often called peptidomimetics from now onwards, and peptides and peptidomimetics are used interchangeably. The present invention also describes that when peptides are made by combination of the cationic monomers of the invention and of natural amino acids/analogues, such hybrid peptides have cationic charge (at least one) on the backbone and exhibit potent biological activities. They are useful in a variety of commercial applications including antimicrobial activity, cell penetration and drug delivery.

### Thiourea Monomer Synthesis:

Another aspect of the present invention covers the method/s of synthesis of monomers described in the present invention. The present inventor has shown that the monomers described above could be synthesized in the laboratory. Few examples of compounds obtained from the generalized scheme for synthesis of the monomers are shown, but are not limited to in Table 2.

### C. COMPOSITION FOR ADMINISTRATION OF THE COMPOUNDS

In an embodiment, the present invention discloses compositions comprising the peptide, oligomers, and oligomer-peptides and pharmaceutically acceptable excipients. The composition of the present invention may be delivered/administered orally (tablets or capsules, solutions, or suspensions), or parenterally (injectables) or topically (ointment, cream, spray, bandages or powder) and intramammary preparations. The composition of the present invention can be administered as a tablet, capsule, syrup, etc, wherein the compound is the dose of 0.1 to 100 mg/Kg body weight of the mammal.

The composition of the present invention may be delivered for its pharmaceutical application in a dosage as described below:

### D. UTILITY OF COMPOSITION OF THE PRESENT INVENTION PEPTIDE SYNTHESIS

Another aspect of the present invention describes use of the above listed peptide, oligomer, and oligomer-peptide hybrid compounds. Pharmaceutical compositions comprising peptides, oligomers, oligomer-peptides are discussed in Section C for its utility as entry promoting agent (excluding oligomers), antimicrobial, antifungal agent. For entry promoting agent (drug delivery), the compounds (carriers) are administered as conjugates or noncovalent complexes with the therapeutic molecules (cargo), such as peptides/proteins or to the nucleic acids/analogue or small molecule drugs. For antimicrobial applications, the compounds are administered as such or as composition comprising compounds of the present invention and known antibiotics or antifungals.

### Commercial Applications of the Present Invention

The monomers and oligomers/peptides/oligomer-peptides described in the present invention have several commercial applications. First, the monomers could be used for making several peptides and polymers. Second, the cationic peptide/polymers have a huge number of commercial applications including their use as antimicrobial, anticancer, anti-inflammatory, cell penetrating and drug delivery agents. Some of the applications of the present invention are described below:

Antimicrobial applications: The present inventor has invented a new method of making molecules with guanidine cationic moieties in the backbone. The molecules described in the present invention could be used as antimicrobial molecules to treat microbial infections (bacterial, and fungal infections) caused by both extracellular and intracellular microbes. Moreover, these molecules are synthesized step-by-step in a controlled manner. Also, the molecular weight and composition is predetermined, and kept constant from batch-to-batch synthesis. The novel method described in the present invention allows to produce uniform product from batch to batch. This allows easy use of these molecules for in-vivo use.

The present invention could be used to inhibit growth and control of microbes. The antimicrobial agents of the present invention are effective in a variety of commercial applications. These peptidomimetics/polymers have direct antimicrobial activity. They are effective in slowing the growth of a microbe as well as killing a microbe (such as, bacteria and fungi/yeast). They is used directly as antimicrobial molecules. To enhance their activity, in some instance, they is used in conjunction with calcium and magnesium chelator, such as, EDTA, EGTA, etc. In some instance, these peptidomimetics enhance activity of the existing antimicrobials (such as antibiotics/antimycotics) by several mechanisms. First, such combination (peptidomimetics+existing antimicrobials, with or without calcium/magnesium chelator, such as, EDTA or EGTA) act as synergistic formulations. Second, these peptidomimetics form nanoparticles with existing antimicrobials and promote their improved entry into a microbe. Third, they inhibit efflux of existing antimicrobials; thus, increasing the concentration of antimicrobial. Fourth, these peptidomimetic molecules enhance the potency of existing antimicrobials by creating pores in the microbial membrane, thus, facilitating improved entry of antimicrobials. Thus, the present invention covers use of cationic backbone peptidomimetics either alone or in synergistic combination with existing antimicrobials or nanoparticle formulations. Apart from their inherent antimicrobial effect, these synthetic peptidomimetics potentiate activity of the existing antimicrobials. The present inventor has surprisingly shown that these peptide analogues facilitate entry of antimicrobials into wide variety of microbes by creating entry pathways in the microbial membrane. The pathways created by enhancer molecules allows improved entry of antimicrobials either together with activity enhancer or after the activity enhancer has created the pathways. Thus, activity enhancers surprisingly increase intracellular concentration of the currently used antimicrobials. By promoting entry of known antimicrobials, the activity enhancer agents potentiate the activity of antimicrobials. By increasing their intracellular concentration, these enhancers indirectly or directly lower their efflux from the microbes. These peptide analogues are also known to target bacterial multidrug resistance system by inhibiting efflux pumps and antibiotic inactivation machinery of the bacterial cells. In some instances, these peptide analogues also form nanoparticles with existing antimicrobials, and improve their cellular entry and efficacy. More importantly, the synergistic combination sensitizes a microbe otherwise ineffective to a known antimicrobial, such as ampicillin/terbinafine (0-100 μg/ml).

The present inventor has invented new ways/methods of formulation having potent synergistic antimicrobial activity, composition and methods for combating microbial growth on a solid/liquid surface or surface of a living animal including humans.

The present invention describes two important aspects of treating topical and systemic microbial infections of humans or animals where current antimicrobials are ineffective. In this embodiment, topical infection collectively refers to microbial (bacteria/mycoplasma, fungi/yeast infections in combinations) infection of skin, ear, eye, foot, teat and udder.

Drug delivery applications: Another aspect of the present invention is related to the compounds and method having entry promoting (broadly referred as drug delivery) properties. Promoting entry of an agent into a cell, the method comprising the step of exposing the cell (eukaryotic or prokaryotic) to the introduced agent (cargo molecules) in the form of complexes comprising the peptides/polymers (entry promoting agent) of the present invention and cargo molecules (introduced agent, in some instance complexes has additional components such as an additional lipids/fatty acids or carbohydrates or proteins to form ternary or quaternary complex to enhance the entry promoting activity). Particularly relates to use of synthetic peptides/polymers described in the present invention as entry promoting agents for delivery of proteins, polypeptides and analogues; nucleic acids and analogues; small molecules (often referred to as drugs, including antimicrobials) with applications in research or medicinal/therapeutics or prophylactics or imaging agents or agents used in research. The present inventor has shown that the individual monomers do not possess cell penetrating properties (except when the terminal amino group is modified to guanidine/analogue or analogue and the other end is conjugated to the cargo), but when they are joined together (more than one, up to 5,000 amino acid residues) they possess potent cell penetrating properties (enter both eukaryotes and prokaryotes; both vertebrates and invertebrate cells; including animal cells, fish cells, prawn cells, bacteria, fungi, yeast, parasites/protozoa, insect and plant cells). They are able to deliver a variety of cargo molecules either as a covalent conjugate or non-covalent mixture (often involving formation of nanoparticles). The entry promoting agents in the present invention is used for delivery into both eukaryotic and prokaryotic cells either in-vitro or in-vivo. The introduced agent (usually called as cargo molecule) may typically be or comprise a bioactive compound (such as, those used in prophylactic or therapeutic applications or diagnostic/imaging probes), proteins, polypeptides, peptides and analogues, small molecules (often referred to as drugs, including antimicrobials), nucleic acids and analogues and synthetic compounds, analogues and derivatives thereof. The method involves exposing a cell (eukaryotic or prokaryotic) to a combination of entry promoting peptides and introduced agents.

In the present invention, entry promoting agents are used to covalently conjugate the introduced molecules (cargo molecules). The present inventor has shown that when an introduced agent (cargo molecules: drug/small molecule/diagnostic probes/peptides, nucleic acids and nucleic acids analogues including locked nucleic acids, morpholino oligonucleotides, phosphorothioates, etc.) when conjugated to the cationic backbone oligomers described in the present invention, they enter very efficiently into both eukaryotic and prokaryotic cells (including animal cells, human cells, insect cells, bacteria, fungi, yeast and protozoa). Alternatively, these cationic backbone peptides are added to the above listed class of diagnostic and therapeutic molecules post synthesis.

Compound or composition of peptides, oligomer-peptides in Section C its utility as a carrier for other molecules into eukaryote and prokaryote cells, wherein the carrier is used up to 1000 fold molar excess of the cargo molecules (peptide, protein, pDNA, oligonucleotides, small molecules)

Use of monomer in synthesis of peptides: Another aspect of the present invention describes the use of the monomers of the present invention for making any peptides. Such peptides will have at least one of the cationic backbones bearing guanidine residue described in this invention. The present invention also describes the novel methods to make hybrid peptide-oligomers with many commercial applications.

Antimicrobial formulations: Another aspect of the present invention is related to the compositions, formulations and methods having microbicidal and biostatic properties, and is particularly useful as anti-bacterial and antifungal agents. The peptides/polymers of the present invention have inherently antimicrobial activities. Also, the present inventor has shown that these molecules show potent synergistic activity when used in combination with existing antimicrobials (antibiotics or antifungals agents) and activity enhancer molecules (polymeric amine, imine, guanidine or biguanide or epsilon-polylysine or cationic carbohydrate-based system, such as, chitosan and derivatives, amino dextrans, chitosan-biguanide, chitosan-guanidine). Another aspect of the invention describes a synergistic combination of cationic molecules of the invention with polymeric guanidine/biguanide, chlorhexidine, epsilon-polylysine and EDTA, and or herbal extracts containing antimicrobial activity and such formulations show synergistic activity and are useful as anti-bacterial, antifungal agents. It also relates to the method of application. Further aspect of the invention relates to the compositions and methods for inhibiting growth of microbial populations on solid/liquid surfaces. Synergistic compositions and methods for the prevention or treatment of mastitis in dairy animals (cow, buffalo, goat, camel, yak and sheep) or topical microbial infections (skin and foot infections), eye and ear infections or systemic infections in humans and animals.

Compound or composition of peptides, oligomers, oligomer-peptides for its utility as antimicrobial and/or antifungal agent further comprises peptides (0.01-300 μg/ml) and known antibiotics/antifungals, such as, ampicillin. Compound or composition of peptides, oligomers, oligomer-peptides in Section A terbinafine (0.01-100 μg/ml), leading to potent synergistic antimicrobial/antifungal activity.

### E. EXAMPLES

The present invention is illustrated by way of examples. The examples are meant only for illustrative purpose and cannot be construed to limit the scope of the invention.

### Examples of Monomers of Formulae 1, 1a, 1b and 1c

The examples of compounds having Formula 1 1a, 1b, 1c are shown below:
i. 101: N-(Benzyloxy carbonyl)-N′-(3-tertbutoxycarbonyl amino propylene) thiourea

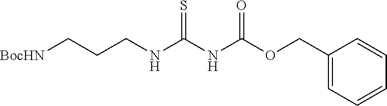 [SMILES: CC(C)(C)OC(=O)NCCCNC(=S)NC(=O)OCc1ccccc1]

ii. 102: N-(Benzyloxy carbonyl)-N′-(4-tertbutoxycarbonyl amino butylene) thiourea

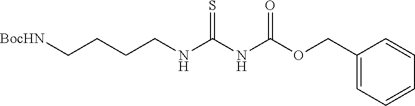 [SMILES: CC(C)(C)OC(=O)NCCCCNC(=S)NC(=O)OCc1ccccc1]

iii. 103: N-(Benzyloxy carbonyl)-N′-(5-tertbutoxycarbonyl amino pentylene) thiourea

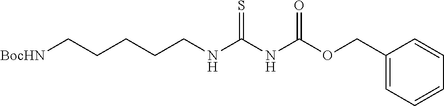 [SMILES: CC(C)(C)OC(=O)NCCCCCNC(=S)NC(=O)OCc1ccccc1]

iv. 104: N-(Benzyloxy carbonyl)-N′-(6-tertbutoxycarbonyl aminohexylene) thiourea

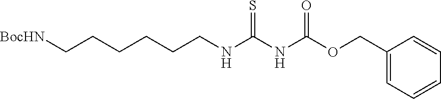 [SMILES: CC(C)(C)OC(=O)NCCCCCCNC(=S)NC(=O)OCc1ccccc1]

V. 105: N-(Benzyloxy carbonyl)-N′-(7-tertbutoxycarbonyl amino heptylene) thiourea

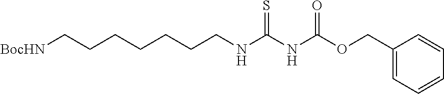 [SMILES: CC(C)(C)OC(=O)NCCCCCCCNC(=S)NC(=O)OCc1ccccc1]

vi. 106: N-(Benzyloxy carbonyl)-N′-(8-tertbutoxycarbonyl amino octylene) thiourea

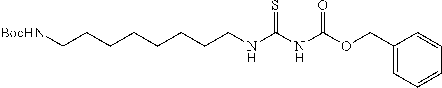 [SMILES: CC(C)(C)OC(=O)NCCCCCCCCNC(=S)NC(=O)OCc1ccccc1]

vii. 107: N-(-Fluorenyl methoxy carbonyl)-N′-(3-tertbutoxycarbonyl amino propylene) thiourea

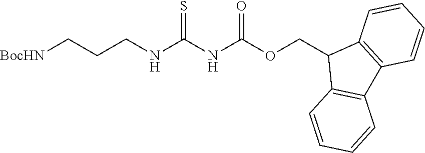 [SMILES: CC(C)(C)OC(=O)NCCCNC(=S)NC(=O)OCC1c2ccccc2-c2ccccc21]

viii. 108: N-(9-Fluorenyl methoxy carbonyl)-N′-(4-tertbutoxycarbonyl amino butylene) thiourea

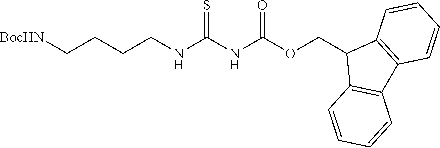 [SMILES: CC(C)(C)OC(=O)NCCCCNC(=S)NC(=O)OCC1c2ccccc2-c2ccccc21]

ix. 109: N-(9-Fluorenyl methoxy carbonyl)-N′-(5-tertbutoxycarbonyl amino pentylene) thiourea

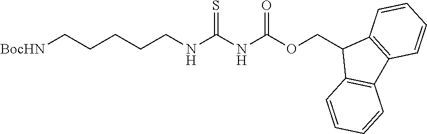 [SMILES: CC(C)(C)OC(=O)NCCCCCNC(=S)NC(=O)OCC1c2ccccc2-c2ccccc21]

x. 110: N-(9-Fluorenyl methoxy carbonyl)-N′-(6-tertbutoxycarbonyl aminohexylene) thiourea

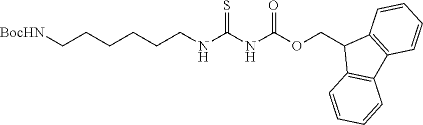 [SMILES: CC(C)(C)OC(=O)NCCCCCCNC(=S)NC(=O)OCC1c2ccccc2-c2ccccc21]

xi. 111: N-(9-Fluorenyl methoxy carbonyl)-N′-(7-tertbutoxycarbonyl amino heptylene) thiourea

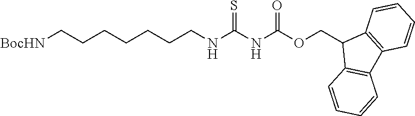 [SMILES: CC(C)(C)OC(=O)NCCCCCCCNC(=S)NC(=O)OCC1c2ccccc2-c2ccccc21]

xii. 112: N-(9-Fluorenyl methoxy carbonyl)-N′-(8-tertbutoxycarbonyl amino octylene) thiourea

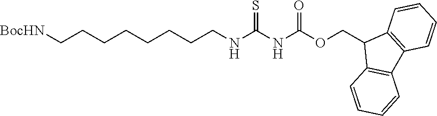 [SMILES: CC(C)(C)OC(=O)NCCCCCCCCNC(=S)NC(=O)OCC1c2ccccc2-c2ccccc21]

xiii. 113: N-(tertbutoxy carbonyl)-N′-(3-fluorenylmethoxycarbonyl amino propyl) thiourea

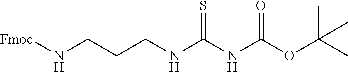

xiv. 114: N-(tertbutoxy carbonyl)-N′-(4-fluorenylmethoxycarbonyl amino butyl) thiourea

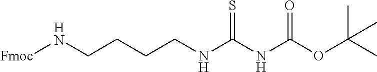

xv. 115: N-(tertbutoxy carbonyl)-N′-(5-fluorenylmethoxycarbonyl amino pentyl) thiourea

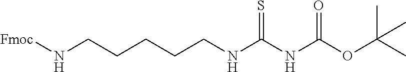

xvi. 116: N-(tertbutoxy carbonyl)-N′-(6-fluorenylmethoxycarbonyl amino hexyl) thiourea;

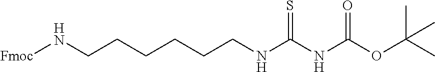

xvii. 117: N-(tertbutoxy carbonyl)-N′-(7-fluorenylmethoxycarbonyl amino heptyl) thiourea

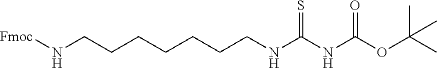

xviii. 118: N-(tertbutoxy carbonyl)-N′-(8-fluorenylmethoxycarbonyl amino octyl) thiourea

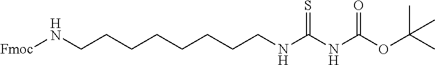

xix. 119:4-fluorenylmethoxycarbonyl amino-N-(4-(3-tertbutyloxycarbonyl thioureido)butyl) butanamide

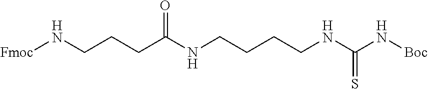

xx. 120:3-fluorenylmethoxycarbonyl amino-N-(4-(3-tertbutyloxycarbonyl thioureido)butyl) propenamide

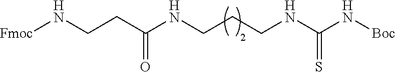

xxi. 121:3-fluorenylmethoxycarbonyl amino-N-(5-(3-tertbutyloxycarbonyl thioureido) pentyl) propenamide

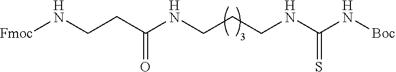

xxii. 122:3-fluorenylmethoxycarbonyl amino-N-(6-(3-tertbutyloxycarbonyl thioureido) hexyl) propenamide

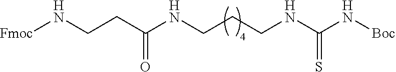

xxiii. 123:6-fluorenylmethoxycarbonyl amino-N-(6-(3-tertbutyloxycarbonyl thioureido) hexyl) hexanamide

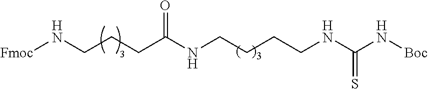

xxiv. 124:4-fluorenylmethoxycarbonyl amino-N-(5-(3-tertbutyloxycarbonyl thioureido) pentyl) butanamide

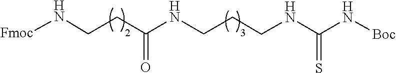

xxv. 125:4-fluorenylmethoxycarbonyl amino-N-(6-(3-tertbutyloxycarbonyl thioureido) hexyl) butanamide

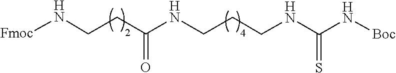

xxvi. 126:6-tertbutyloxycarbonyl amino-N-(6-(3-fluorenylmethoxycarbonyl thioureido) hexyl) hexanamide

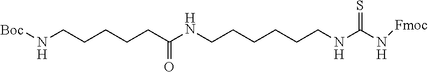

xxvii. 127:6-(3-fluorenylmethoxycarbonyl thioureido)-N-(6-tertbutyloxycarbonylamino) hexyl) hexanamide

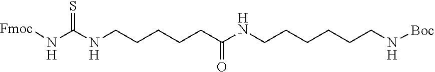

Example 1 of compound from Formula 1a: N-(9-Fluorenyl methoxy carbonyl)-N′-(6-tertbutoxycarbonyl aminohexylene) thiourea was synthesized with the structural Formula

This is substantiated by ESI-MFE; [M+1]⁺=496.24, Expected mass: 496.24.

¹H-NMR (400 MHZ, CHCl₃-D): δ 7.79 (d, 2H), 7.54 (d, 2H), 7.26-7.4 (m, 4H) 4.3 (d, 2H) 4.23 (m, 1H) 3.5 (m, 2H), 2.9 (m, 2H), 1.15-1.6 (m, 17H).

Example 2 of compound from Formula 1a: N-(9-Fluorenyl methoxy carbonyl)-N′-(3-tertbutoxycarbonyl amino propylene) thiourea was synthesized with the structural Formula

This is substantiated with 1H-NMR (400 MHZ, CHCl₃-D): δ 7.769 (d, 2H), 7.54 (d, 2H), 7.2-7.3 (m, 4H) 4.48 (d, 2H) 4.23 (m, 1H) 3.7 (m, 2H), 3.1 (m, 2H), 1.8 (m, 2H), 1.45 (9H).

Example 3 of compound from Formula 1a: N-(9-Fluorenyl methoxy carbonyl)-N′-(4-tertbutoxycarbonyl amino butylene) thiourea was synthesized with the structural Formula

This is substantiated with 1H-NMR (400 MHz, CHCl₃-D): δ 7.79 (d, 2H), 7.54 (d, 2H), 7.26-7.4 (m, 4H) 4.48 (d, 2H) 4.23 (m, 1H) 3.7 (m, 2H), 3.1 (m, 2H), 1.5-1.8 (m, 4H), 1.43 (9H).

Example 4 of compound from Formula 1a: N-(Benzyloxy carbonyl)-N′-(6-tertbutoxycarbonyl aminohexylene) thiourea with the structural formula was synthesized as

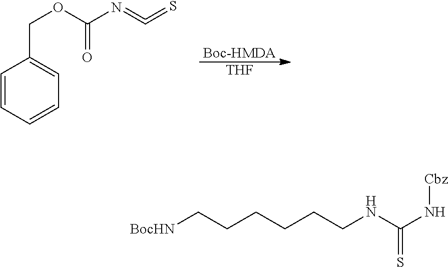

This is substantiated by LC-MS: [m/z]⁺=409.20, Molecular Formula=C₂₀H₃₁N₃O₄S; Exact mass: 409.20+1H=410.20.

Example 5 of compound from Formula 1a: N-(Benzyloxy carbonyl)-N′-(4-tertbutoxycarbonyl amino butylene) thiourea was synthesized as

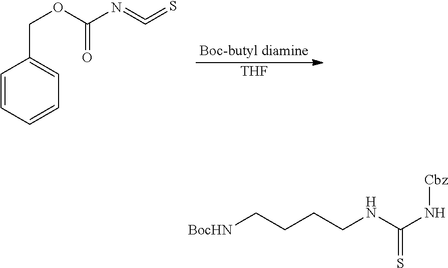 [SMILES: O=C(N=C=S)OCc1ccccc1 ; "Boc-butyl diamine" ; CC(C)(C)OC(=O)NCCCCNC(=S)NC(=O)OCc1ccccc1 ; "THF"]

This is substantiated by 1H-NMR (400 MHZ, CHCl₃-D): δ 7.34-7.36 (m, 5H), 5.16 (s, 2H), 3.6 (m, 2H), 3.1 (m, 2H), 1.6 (m, 2H), 1.6 (m, 2H), 1.5 (m, 2H), 1.47 (s, 9H).

Example 6 of compound from Formula 1a: N-(Benzyloxy carbonyl)-N′-(3-tertbutoxycarbonyl amino propylene) thiourea was synthesized with the structural formula

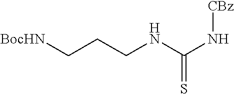

This is substantiated by 1H-NMR (400 MHZ, CHCl₃-D): δ 7.34-7.36 (m, 5H), 5.16 (s, 2H), 3.6 (m, 2H), 3.1 (m, 2H), 1.9 (m, 2H), 1.47 (s, 9H)

Example 7 of compound from Formula 1a: N-(tertbutoxy carbonyl)-N′-(6-fluorenylmethoxycarbonyl amino hexyl) thiourea was synthesized using structural formula

This is substantiated by 1H-NMR (400 MHZ, CHCl₃-D): δ 7.29-7.77 (m, 8H), 4.38-4.65 (m, 3H), 3.09-3.27 (m, 4H), 1.25-1.485 (17.0H).

Example 8 of compound from Formula 1c: 4-fluorenylmethoxycarbonyl amino-N-(4-(3-tertbutyloxycarbonyl thioureido)butyl) butanamide was synthesized with general formula

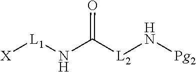

where X is, L1 is, L2 is and Pg₂ is Fmoc. On substitution, the structural formula for 4-fluorenylmethoxycarbonyl amino-N-(4-(3-tertbutyloxycarbonyl thioureido)butyl) butanamide is

This is substantiated by ESI-MS; [m/z]⁺=554.26, Molecular weight: 554.71; [M+−101 (Boc)]=453.34; Expected mass: 554.26; Molecular formula: C₂₉H₃₈N₄O₅S.

Example 9 of compound from Formula 1c: 6-tertbutyloxycarbonyl amino-N-(6-(3-fluorenylmethoxycarbonyl thioureido) hexyl) hexanamide

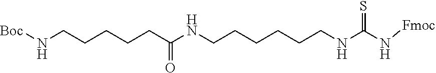

This is substantiated by ESI-MS: Expected mass: 610.32, Observed mass [m/z]⁺: 611.32 (M+H)⁺ Chemical Formula: C₃₃H₄₆N₄O₅S

### Examples of Cationic Guanidine Based Peptide, Oligomer and Oligomer-Peptide Hybrid Compound of Formula 2

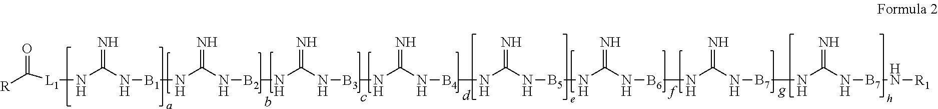

Example 1: R═NH₂, L1=—(CH₂)₅—, B1=B2=B3=B4=B5=—(CH₂)₆—, R1=—H,
a, b, c, d, e=1, f, g, h=0

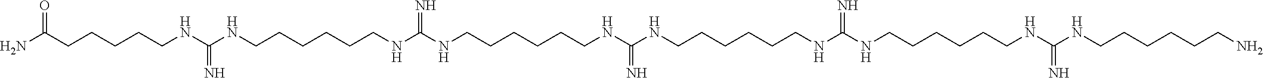 [SMILES: N=C(NCCCCCCN)NCCCCCCNC(=N)NCCCCCCNC(=N)NCCCCCCNC(=N)NCCCCCCNC(=N)NCCCCCC(N)=O]

Expected mass: 835.74; Observed mass: 836.6 (M+H)⁺

Example 2: R═NH₂, L1=—(CH₂)₅, B1=B2=B3=B4=B5=

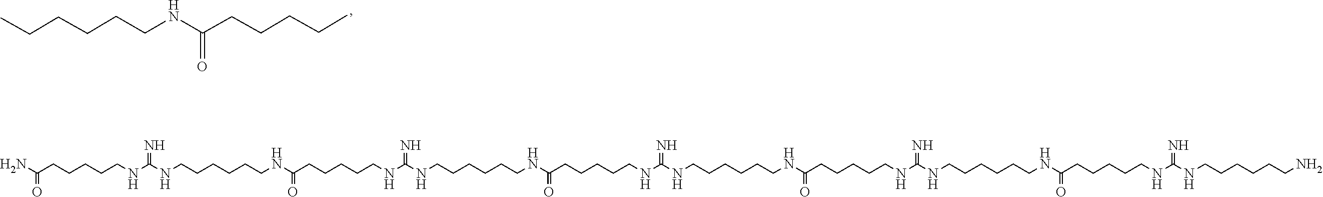 [SMILES: CCCCCCNC(=O)CCCCC ; N=C(NCCCCCCNC(=O)CCCCCNC(=N)NCCCCCCNC(=O)CCCCCNC(=N)NCCCCCCNC(=O)CCCCCNC(=N)NCCCCCCNC(=O)CCCCCNC(=N)NCCCCCCN)NCCCCCC(N)=O]

a, b, c, d, e=1, f, g, h=0, R1=—H
Molecular weight: 1288.92; Observed mass: 1291.96 (M+3H)⁺

Example 3: When the compound is synthesized using monomer of Formula 1a, wherein R═NH₂, L1=—(CH₂)₅—, B1=B2=B3=B4=B5=—(CH₂)₆—, R1=—H,
a, b, c, d, e=1, f, g, h=0 the resulting compound is Oligomer (5-mer)

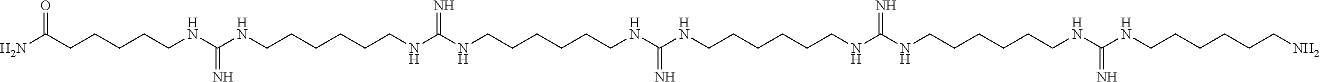 [SMILES: N=C(NCCCCCCN)NCCCCCCNC(=N)NCCCCCCNC(=N)NCCCCCCNC(=N)NCCCCCCNC(=N)NCCCCCC(N)=O]

Expected mass: 835.74; Observed mass: 836.6 (M+H)⁺

Example 4: When the compound is synthesized using monomer of Formula 1a, wherein R═NH₂, L1=—(CH₂)₅—, B1=B2=B3=B4=B5=B6=B7=—(CH₂)₆—, R1=—H, a, b, c, d, e, f, g=1, h=3 the resulting compound is Oligomer (9-mer)

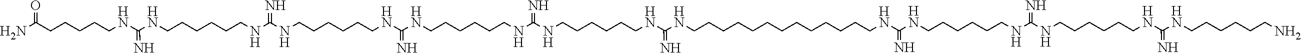 [SMILES: N=C(NCCCCCCN)NCCCCCCNC(=N)NCCCCCCNC(=N)NCCCCCCCCCCCCCNC(=N)NCCCCCCNC(=N)NCCCCCCNC(=N)NCCCCCCNC(=N)NCCCCCCNC(=N)NCCCCCC(N)=O]

This is substantiated with MALDI-TOF, Expected mass: 1400.25, Observed mass: 1400.932

Example 5: When the compound is synthesized using monomer of Formulae 1a, 1b or 1c, wherein R═NH₂, L1=—(CH₂)₅, B1=B2=B3=B4=

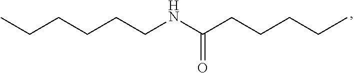

B5=(CH₂)₆,
a, b, c, d, e=1, f, g, h=0, R1=—H the resulting compound is peptide

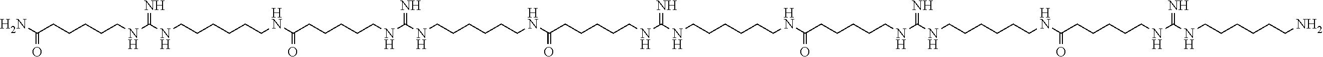 [SMILES: N=C(NCCCCCCNC(=O)CCCCCNC(=N)NCCCCCCNC(=O)CCCCCNC(=N)NCCCCCCNC(=O)CCCCCNC(=N)NCCCCCCNC(=O)CCCCCNC(=N)NCCCCCCN)NCCCCCC(N)=O]

Molecular Weight: 1288.92; Observed mass: 1291.96 (M+3H)⁺

Example 6: When the compound is synthesized using monomer of Formulae 1a, 1b or 1c, wherein R═NH₂, L1=−(CH₂)₃, B1=

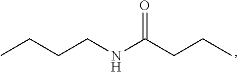

B2=(CH₂)₄, a, b=1, R1=H, the wherein R═NH₂, L1=—(CH₂)₃, B1=resulting compound is

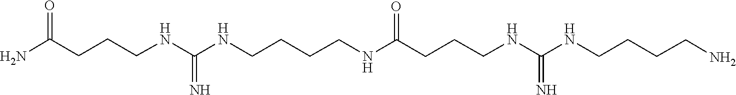 [SMILES: N=C(NCCCCNC(=O)CCCNC(=N)NCCCCN)NCCCC(N)=O]

This is substantiated by ESI-MS. Expected mass: 413.32; Observed mass: 452.91 (M+K⁺)

Example 7: When the compound is synthesized using monomer of Formulae 1a, 1b or 1c, wherein R═NH₂, L1=−(CH₂)₃,

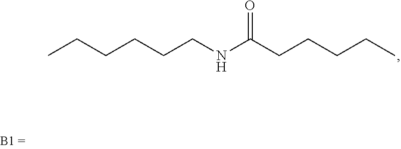

B2=(CH₂)₆, R1=H

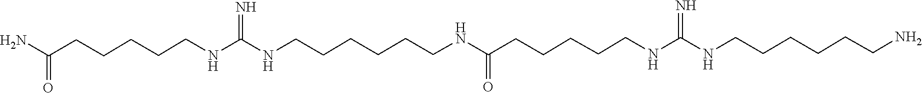 [SMILES: N=C(NCCCCCCNC(=O)CCCCCNC(=N)NCCCCCCN)NCCCCCC(N)=O]

This is substantiated by ESI-MS. Expected mass: 525.45; Observed mass: 565.72 (M+K⁺)

### Examples of Hetero-Oligomers of Formula 2

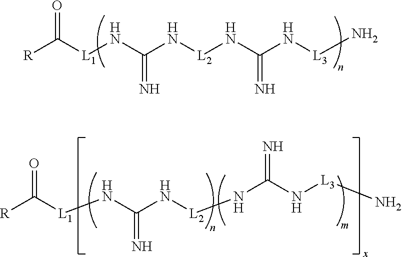

where R is —NH₂, —OH
L1, L2 and L3 are same as defined for Formula 1,
n, m is independently selected from an integer from 1 to 100 which is same or different.
x is also an integer from 1 to 100.
C-3-C-6 (1-1) (n mer)

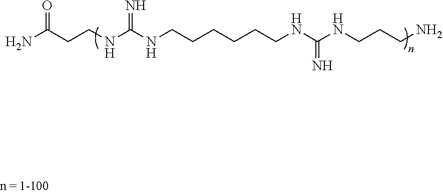

C3-C6 (2-2) (n mer)

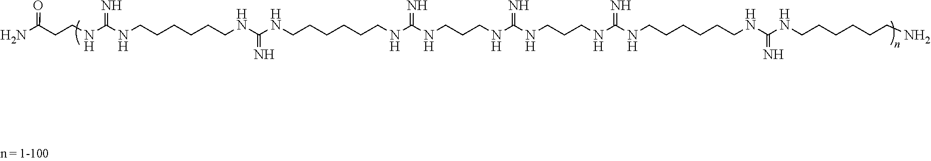

C3-C6 (3-3) (n mer)

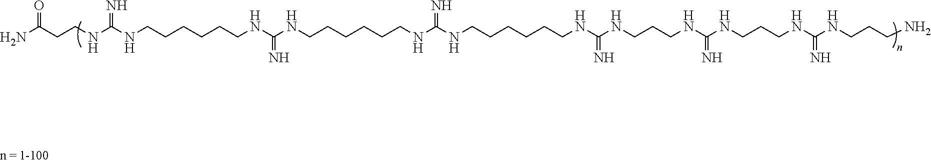

C4-C6 (1-1) (n mer)

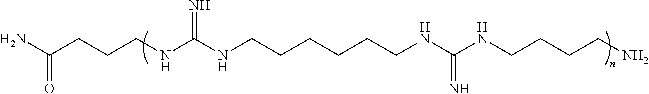

C4-C6 (3-3) (n mer)

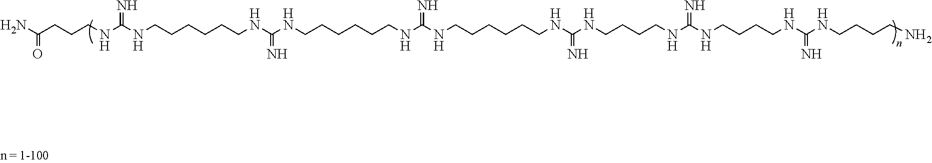

C4-C6 (2-2) (n mer)

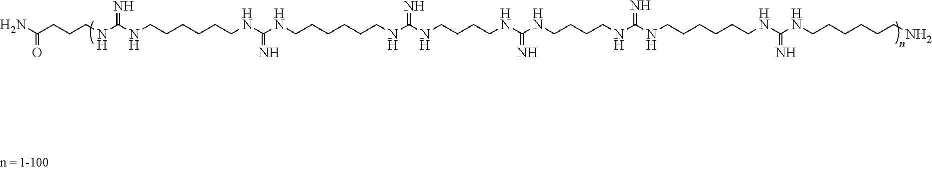

### Example of Heteropolymer Consist of Amide Linker and Alkyl Chain Linker of Formula 2

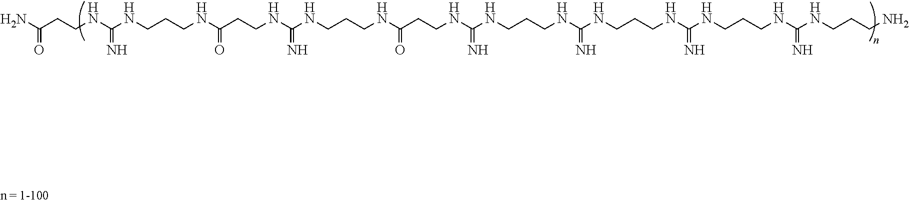

### Examples of Oligoguanidine Peptides from Formula 2

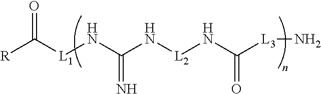

wherein R, L1, L2, L3 same as defined above

For example, when L1=L2=L3=(CH₂)₂ the resulting peptide is shown below

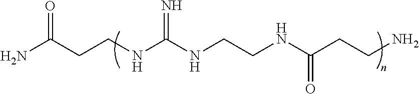

### Other Examples of Formula 2

Other examples, but not limited are shown below,

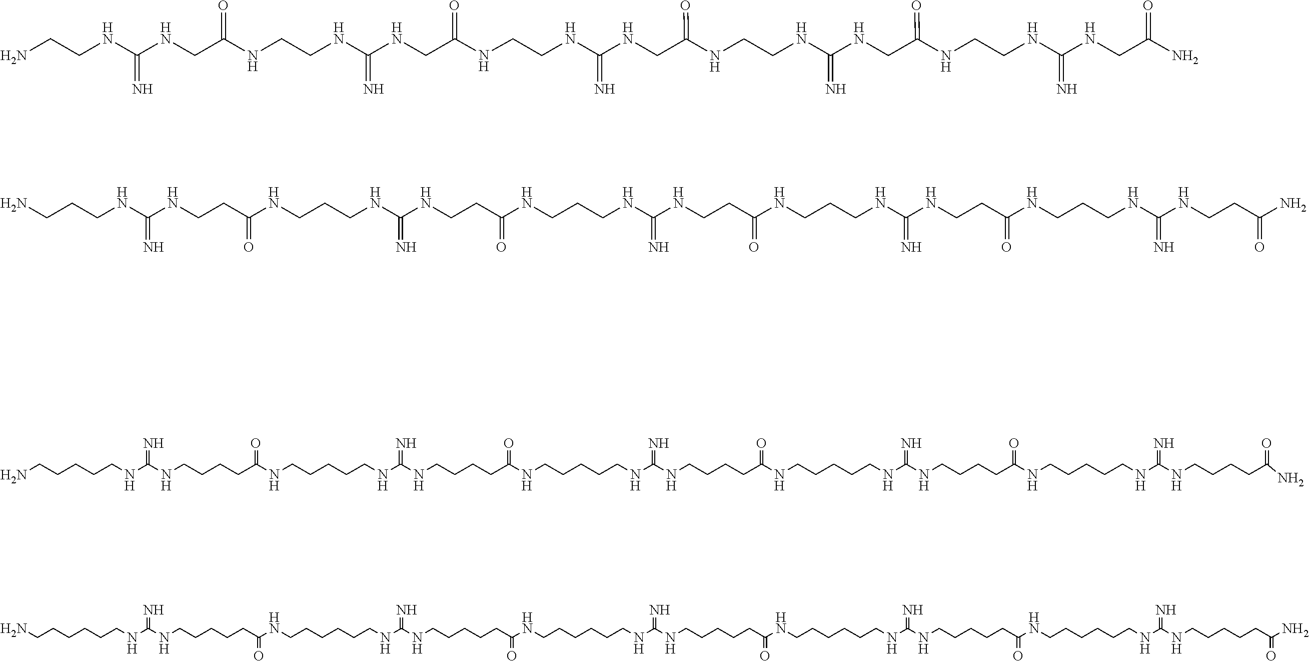 [SMILES: N=C(NCCNC(=O)CNC(=N)NCCNC(=O)CNC(=N)NCCNC(=O)CNC(=N)NCCNC(=O)CNC(=N)NCCN)NCC(N)=O ; N=C(NCCCNC(=O)CCNC(=N)NCCCNC(=O)CCNC(=N)NCCCNC(=O)CCNC(=N)NCCCNC(=O)CCNC(=N)NCCCN)NCCC(N)=O ; N=C(NCCCCCNC(=O)CCCCNC(=N)NCCCCCNC(=O)CCCCNC(=N)NCCCCCNC(=O)CCCCNC(=N)NCCCCCNC(=O)CCCCNC(=N)NCCCCCN)NCCCCC(N)=O ; N=C(NCCCCCCNC(=O)CCCCCNC(=N)NCCCCCCNC(=O)CCCCCNC(=N)NCCCCCCNC(=O)CCCCCNC(=N)NCCCCCCNC(=O)CCCCCNC(=N)NCCCCCCN)NCCCCCC(N)=O]

### Examples for the Homo-Oligomer Compounds of Formula 2

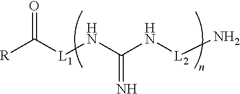

where R: —NH₂, —OH
L1 is same as defined for Formula 1,

The preferred oligomer-guanidine backbone peptide hybrid of Formula 2 and respective deprotected/cleaved product of the present invention are for example and not limited to:

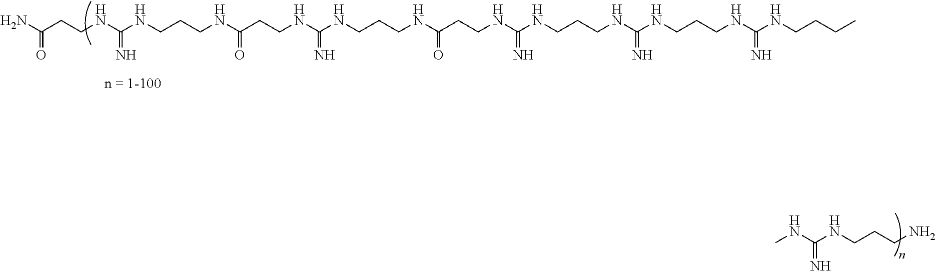

### Example of Cationic Guanidine Based Oligomer-Peptide Hybrid Compound of Formula 3

Example 1:18-mer of oligomer-peptide hybrid, where the compound is synthesized on a lysine two arm MBHA resin support.

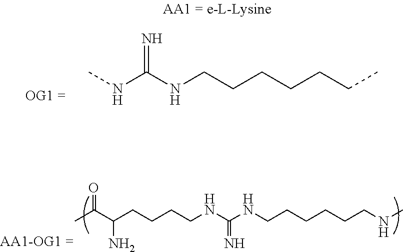

The resulting structure

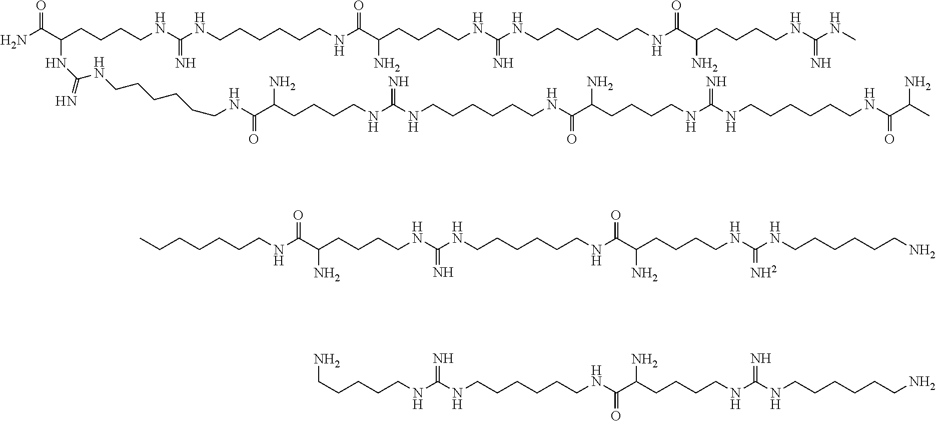

This is substantiated using MALDI-TOF
Calculated mol. wt.: 2581.15
Observed mol. wt.: 2605.63 (M+Na⁺)

Example 2: Synthesis of C3-C4 6mer peptide, with structural formula

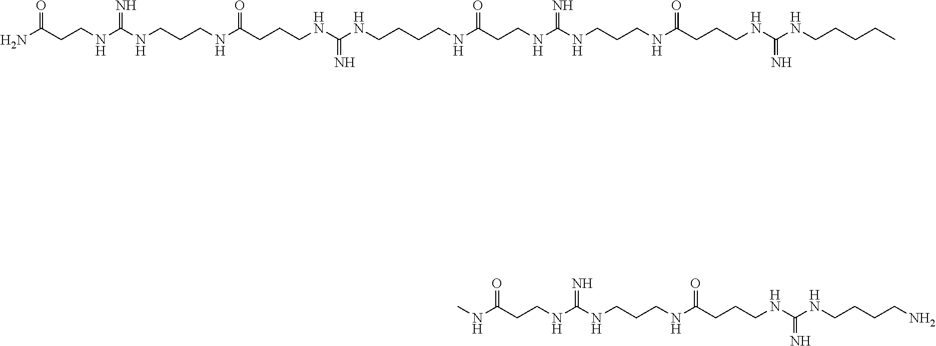

where R═NH₂; L1=—(CH₂)₂; B1, B3, B5=; a-f=1; B2, B4, B6=;
   R1=H.

This is substantiated by MS; (MH++K⁺)=1163.99, Expected mass: 1122.

Example 3:5-mer with structural formula
where R═NH₂; L1=—(CH₂)₅; B1-B5=; a-e=1; R1=H.

This is substantiated by MS; (M+3H)⁺=1291.96; Expected mass: 1288.92; Molecular formula: C₆₅H₁₃₃N₂₁O₅.

The preferred oligomer-peptide hybrid of Formula 3 and respective deprotected/cleaved product of the present invention are for example and not limited to:
when AA1=alanine and OG1=C3-guanidine.

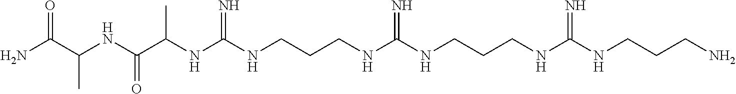 [SMILES: CC(NC(=O)C(C)NC(=N)NCCCNC(=N)NCCCNC(=N)NCCCN)C(N)=O]

when AA1=alpha lysine and OG1=C6-guanidine.

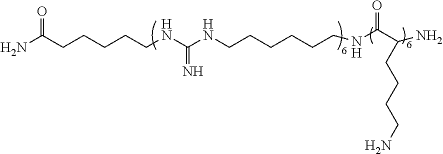

when AA1=alpha lysine and OG1=C3-guanidine.

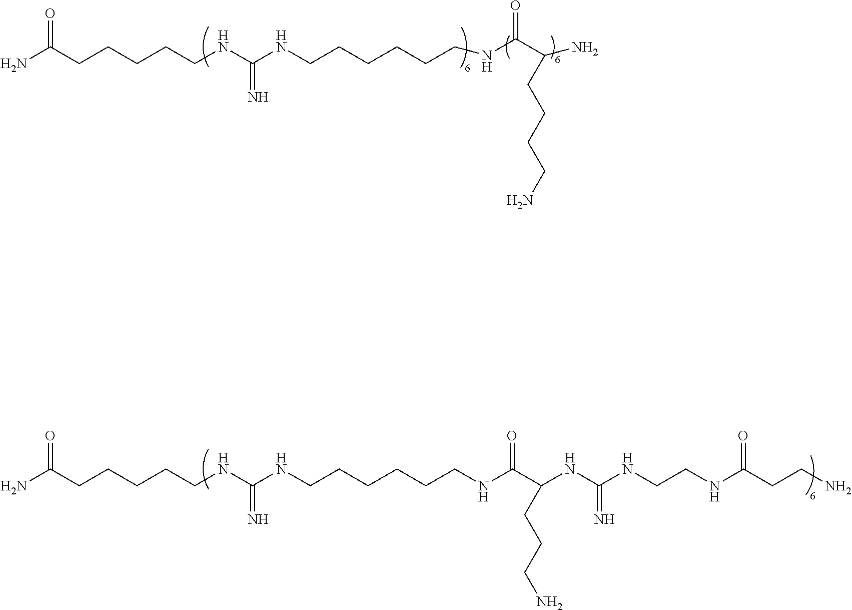

### Example of Resin Bound Structure of Peptide-Oligomer Hybrid of Formula 3

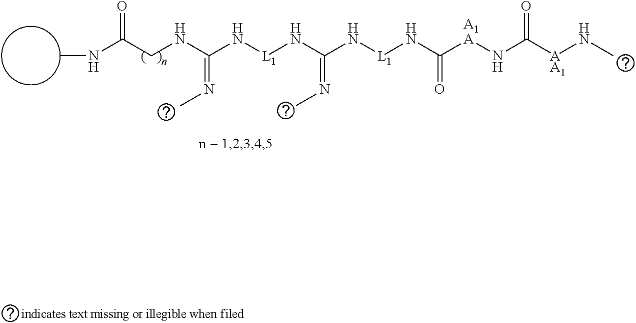

AA1 represented as amino acid residue representing any of the or combination of glycine, alanine, leucine, isoleucine, valine, glutamic acid, aspartic acid, asparagine, glutamine, arginine, histidine, lysine, tryptophan, tyrosine, cysteine, methionine, proline, phenyl alalnine, serine and threonine. AA1 is not limited to natural amino acids, synthetic amino acids are also included.

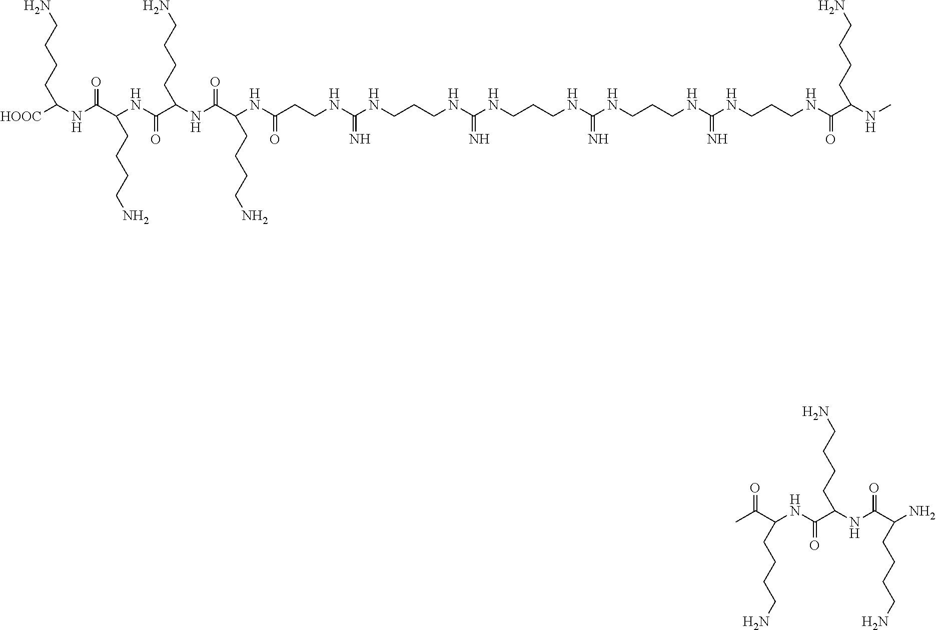

### Example of End Group Modification

Example when L1=(CH₂)₅ and L2=(CH₂)₆, the resulting oliogmer is represented by

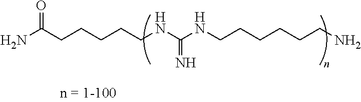

### Example of Cationic Guanidine Based Oligomer-Peptide Hybrid Compound of Formula 3 as Carrier Molecule

Formula 3 is the carrier which facilitate entry through cell membrane of eukaryote and prokaryote cells, AA1 is independently selected from the natural and synthetic amino acid and R1 is defined as end group modification which is extended to drug or cargo, peptide nucleic acid (PNA).

The N-terminus of the oligomer is conjugated to fluorescent labelling compound FITC (Fluorescein isothiocyanate) or to the therapeutic nucleic acid analogue in the manner as shown below to obtain the corresponding conjugated product. The conjugated products are used as diagnostic agent and therapeutic agent respectively.

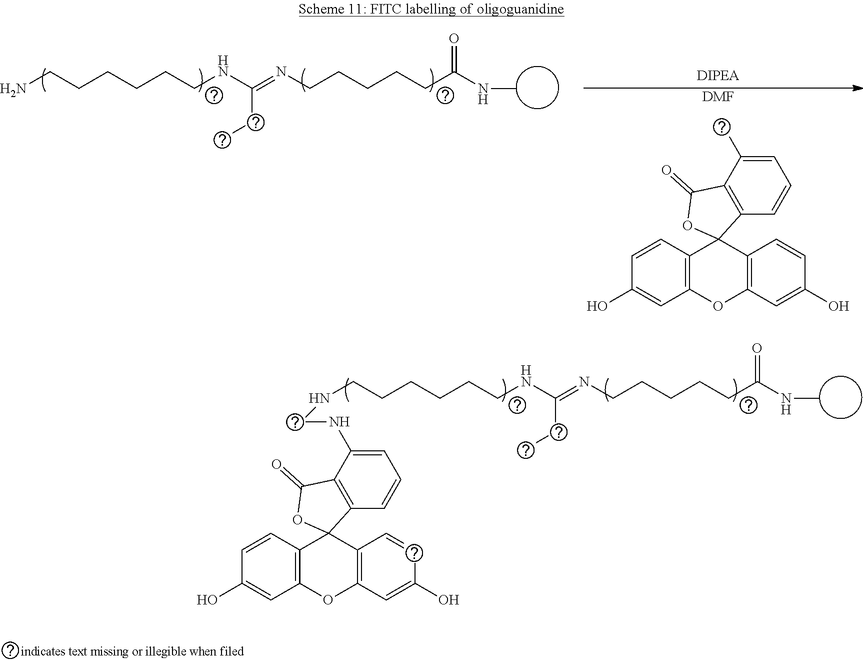

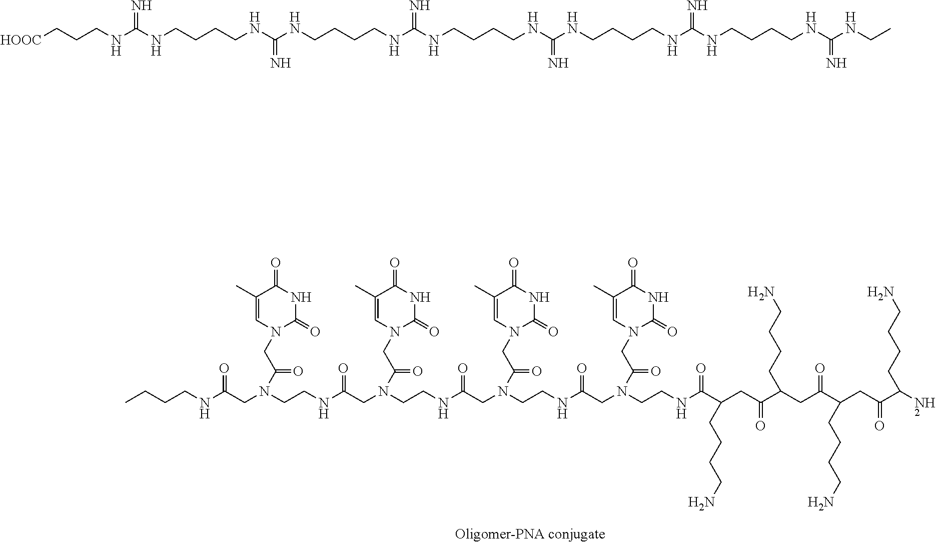

### Examples of Peptides or Oligomer or Peptide-Oligomer Hybrids in the Form of Dendrimers

The present invention also describes synthesis of peptides or oligomer or peptide-oligomer hybrids in the form of dendrimers
2-arm branched 3-arm branched

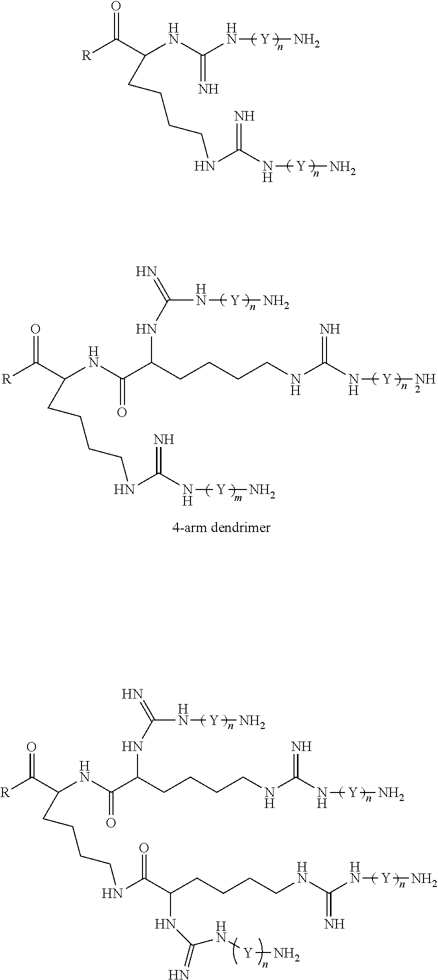

where R is OH or NH₂; Y is selected from -(AA1)ₘ₁-(OG1)ₙ₁-, (OG1)ₘ₁-(AA1)ₙ₁-; -(OG1)ₘ₁-(AA1)ₙ₁-(OG1)ₘ₂-, or -(AA)ₘ₁-(OG1)ₙ₁-(AA1)ₘ₂—, wherein OG1 is oligomer residue from Formula 1 and AA1 is amino-acid residue; n is 1-100.

### Example for Lysine Two-Arm Oligomers Using Monomer from Formula 1

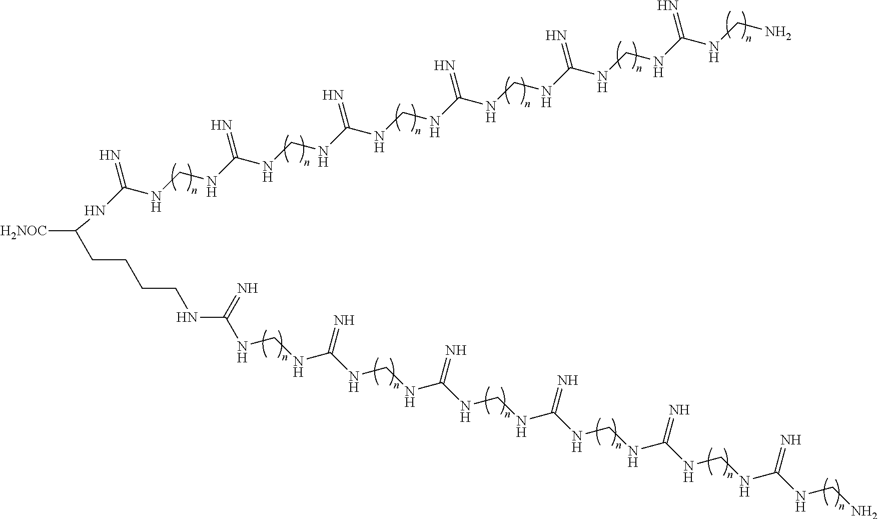

### Example for Three-Arm Oligomer Using Monomer from Formula 1

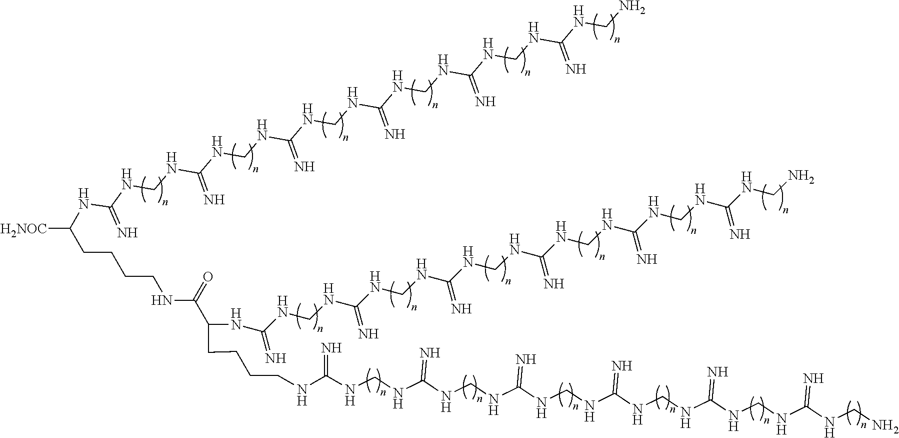

### Example of 4-Arm Dendrimer Using Monomer from Formula 1

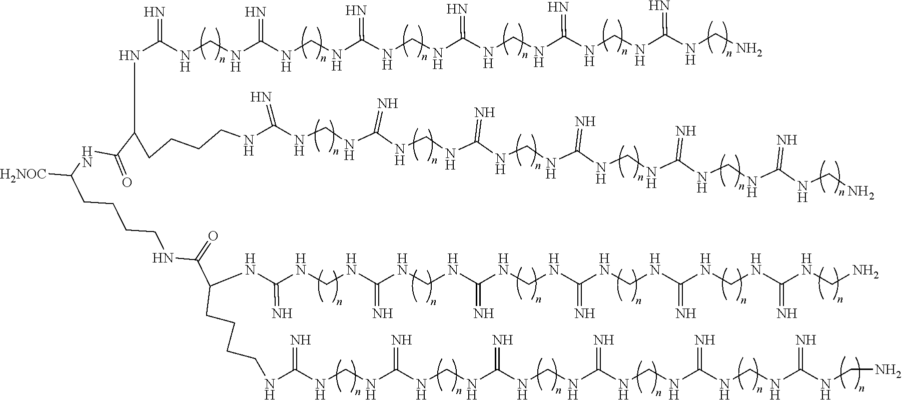

### Testing of Peptide-Oligomers in Antimicrobial Resistance and Drug Delivery Applications Methods Used

Electrophoretic mobility shift assay (EMSA): pDNA and oligonucleotide: For binding studies, EMSA was performed. Briefly, the complexes of oligonucleotide-6FAM/pDNA and peptides, peptide-oligomers were prepared in 20 μl PBS (to achieve a w/w ratio of 0-12), by incubating for 30 minutes at room temperature (25-30° C.). Sample loading dye (final concentration 1×) was then added to the peptide-DNA/oligo-FAM complex. Each sample was then subjected to electrophoresis using 1% agarose gel in 1×-TAE buffer at 100V. DNA bands were visualized by using EtBr. In case of peptide-oligo (FAM labelled), EtBr was not used.

Minimum inhibitory concentration (MIC): Early-log phase cultures of Escherichia coli or Staphylococcus aureus (10⁵ cfu/ml) were incubated with different concentration of test oligomers/peptides/peptide-oligomers (0.50 μg/ml) in a final volume of 200 μl MH broth (MHB), 96 well plates. The plate was incubated in a SpectraMax iD5 reader for 16-18 hours. The bacterial growth was measured by recording absorbance at 600 nm every 5 minutes.

Carrier for peptide, protein, pDNA, oligonucleotides, small molecules: The carrier is used upto 1000 fold molar excess of the cargo molecules (peptide, protein, pDNA, oligonucleotides, small molecules)
i. Peptides, oligomer-peptides and other peptide analogues (0-50 mg/ml of water of PBS) and pDNA (0-50 g/ml of water of PBS)
ii. Peptides, oligomer-peptides and other peptide analogues (0-50 mg/ml of water of PBS) and oligonucleotides/nucleic acid analogues (0-50 μg/ml of water of PBS).
iii. Peptides, oligomer-peptides and other peptide analogues (0-50 mg/ml of water of PBS) and siRNA/miRNA/analogues (0-50 ng/ml of water of PBS).
iv. Peptides, oligomer-peptides and other peptide analogues (0-50 mg/ml of water of PBS) and peptides (0-50 g/ml of water of PBS).
v. Peptides, oligomer-peptides and other peptide analogues (0-50 mg/ml of water of PBS) and proteins (0-50 μg/ml of water of PBS).
vi. Peptides, oligomer-peptides and other peptide analogues (0-50 mg/ml of water of PBS) and small molecules (0-50 μg/ml of water of PBS).
vii. Peptides, oligomer-peptides and other peptide analogues-drug conjugates (0-50 mg/ml)

Oligo delivery: Vero cells were seeded at a density of 10⁹ cells in 24-well plates in DMEM media supplemented with 5% heat inactivated-FBS. Next day, the complexes of oligo-FAM (1 μM) and peptides, peptide-oligomer were prepared in 100 μl PBS and incubated for 30 minutes at room temperature. The complex thus formed was then added to the 24 well plate having Vero cells and kept in 37° C. for 2-3 hours. After incubation the cells were washed with 1×-PBS, 2-3 times. The intracytoplasmic and intranuclear oligo delivery was then monitored by using fluorescent microscope (Nikon eclipse Ti2).

Protein delivery: The complexes of protein (R-phycoerythrin, 0-2 μg) and peptides, peptide-oligomer (0-50 μg) were prepared in 100 μl PBS and incubated for 30 minutes at room temperature. The complex thus formed was then added to the 24 well plate having Vero cells and kept in 37° C. for 2-3 hours. After incubation the cells were washed with 1×-PBS, 2-3 times. The protein delivery was then monitored by using a fluorescent microscope.

DNA delivery: The complexes of plasmid DNA (encoding GFP, 500 ng) and peptides, peptide-oligomer (0-50 μg) were prepared in 100 μl PBS for 30 minutes at room temperature. The complex was then added to a 24 well plate having HEK-293T cells and kept in 37° C. for 48-72 hours. The pDNA delivery was measured by assessing GFP expression using a fluorescent microscope.

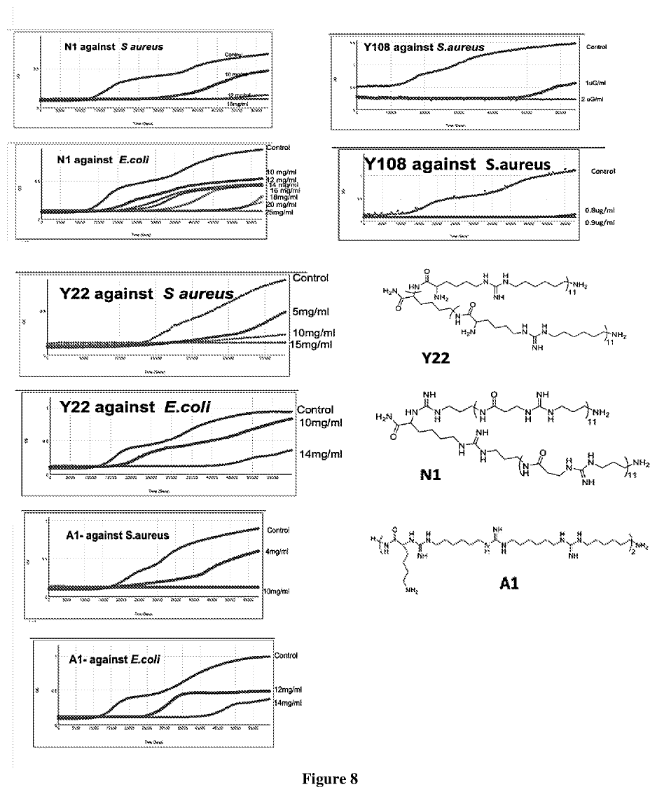

Cell uptake assay: The FITC labelled peptides at varied concentration (0-50 g/ml) were added directly into a 24 well plate having Vero cells, and incubated at 37° C. for 2-3 hours. Then, cells were washed with 1×-PBS, 2-3 times. Similarly, to test uptake into yeast cells, Pichia pastoris culture were treated with FITC labelled peptides for 3 hours. The peptide entry int cells was then observed using fluorescent microscope (FIG. 8).

Minimum inhibitory concentration (MIC) assay: Early log phase culture of either E. coli or S. aureus (10⁵ cfu/ml) were treated with different concentrations of oligomers/peptides/peptide-oligomers (0-5 mg/ml) in 200 μl of MHB in 96 well plates and kept for 16-18 hours at 37° C. The growth was recorded by measuring absorbance at 600 nm every 5 minutes. The concentration which showed complete inhibition of growth was recorded as MIC.

Synergy: MIC experiments were performed as above using a combination of oligomers/peptides/peptide-oligomers (0-1×MIC) and ampicillin (0-50 μg/ml, 0-50×MIC of ampicillin against sensitive strain) using ampicillin resistant S. aureus strain.

MIC-antifungal: The Saccharomyces cerevisiae and Candida albicans were grown on Sabouraud Dextrose Agar at 30° C. for 48 h. Yeast cells were grown in RPMI-1640 media supplemented with 2% glucose. MIC experiments were performed in 96 well plates in a volume of 200 μl, compounds (0-100 μg/ml) and an inoculum of 1-5×10⁵ cfu/m1. The concentration showing complete inhibition is considered MIC.

### Results

The results obtained from testing of peptide-oligomers synthesized in the present invention in antimicrobial resistance and as carrier for other molecules in drug delivery applications in eukaryotic and prokaryotic cells are discussed as follows:

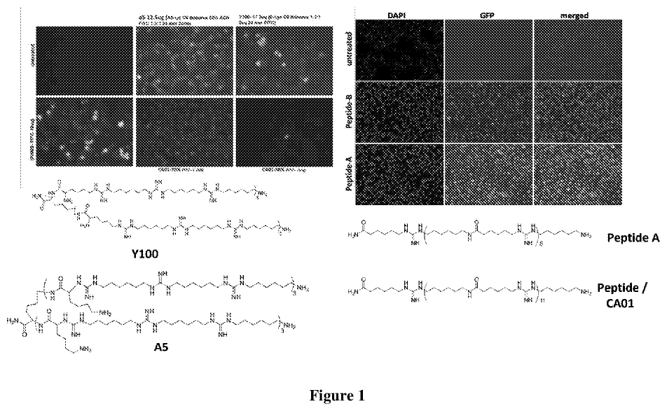

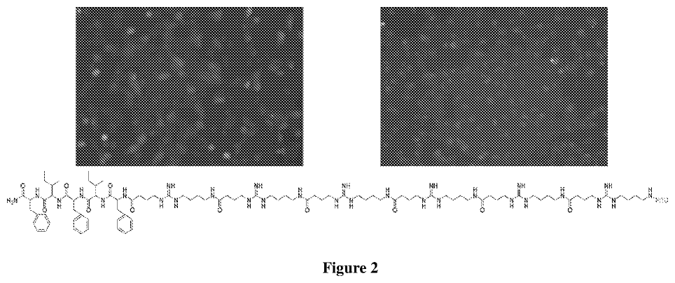

Uptake of peptides into mammalian cells: In order to check the cell penetrating properties of the compounds of the invention, Vero cells were treated with FITC labelled peptides for 2 hours. We noticed that all the peptides tested showed intercellular uptake into >99.99% of the Vero cells in nanomolar-sub micromolar range (A5-FITC, Y100-FITC, CA01-FITC-, CA01-50% FITC-12 μg). The results strongly suggested that the compounds of the present invention have a strong cell penetration property, one of the prerequisites for carrier potential (FIG. 1) Delivery of peptide drug as a conjugate: To check the carrier potential of the compounds of the invention, a tripeptide was conjugated to one of the compounds of the invention and labeled with FITC. An entry into >99.99% cells was noticed at sub micromolar concentration, suggesting that the compounds of the invention have the potential to carry drugs as a conjugate (FIG. 2).

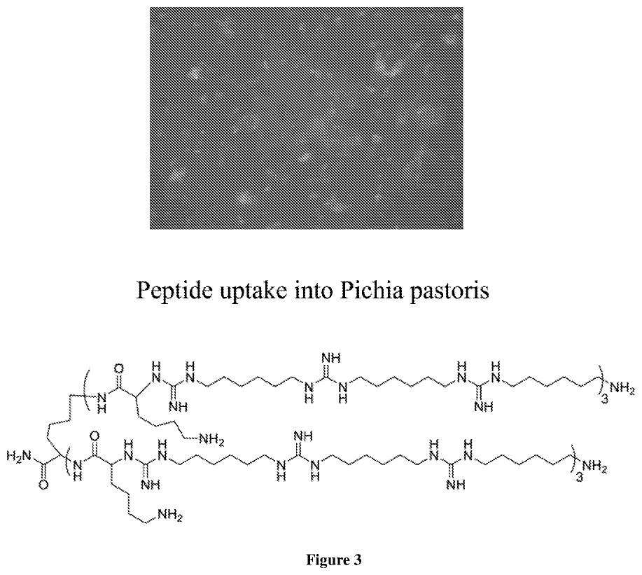

Uptake of peptides into yeast cells: In order to check the cell penetrating properties of the FITC labelled peptides into yeast cells, Pichia pastoris strain was used. As excepted with cell penetration into mammalian cells, the peptides showed similar cell uptake properties into yeast cells as well (FIG. 3), suggesting the compounds of the invention have an entry property into a wide variety of cell types.

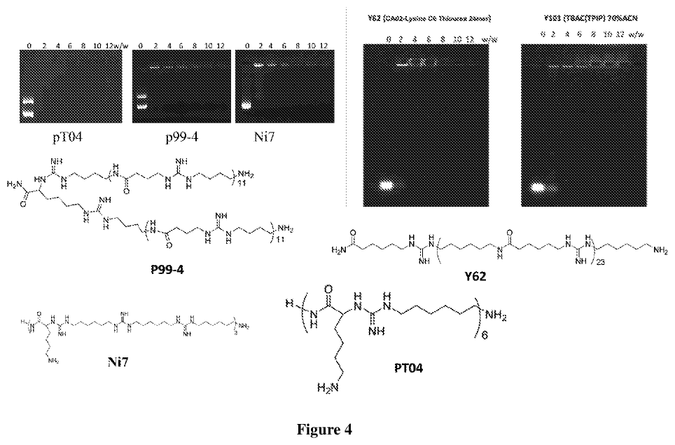

Interaction with pDNA and oligo: In order to check interaction of the compounds with the pDNA/Oligonucleotides EMSA assay was performed. As expected, an interaction was evident by retardation of nucleic acid migration, at a critical concertation complete retardation was noticed where nucleic acid remained in the gel, at higher concentration ethidium bromide access to nucleic acid was observed (no band visible), suggesting encapsulation of the nucleic acid by peptide/analogues, an indication of nanoparticle formation (FIG. 4).

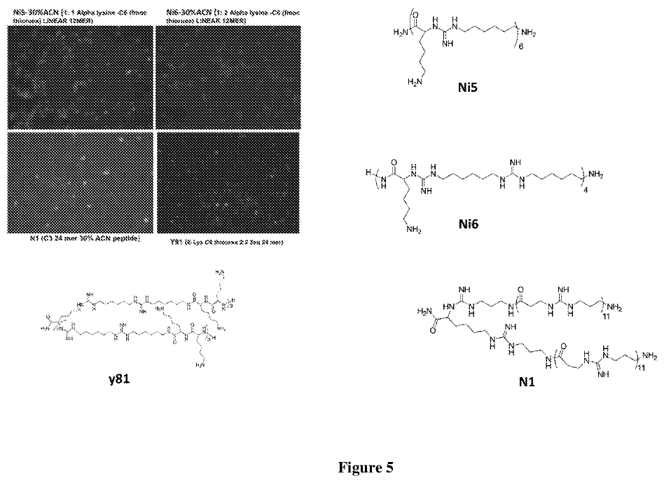

Delivery of oligonucleotides into mammalian cells: To investigate the oligonucleotide delivery efficiency of peptides, Vero cells transfected with FAM labelled oligonucleotide-peptide complex. All different kinds of peptides (guanidine back bone peptides, peptide-oligomer hybrids, guanidine backbone-natural amino acid peptide hybrids) showed clear intra nuclear delivery into more than 40% of the cells (FIG. 5). Suggesting that the compounds of the invention have carrier potential into nucleic acid analogues.

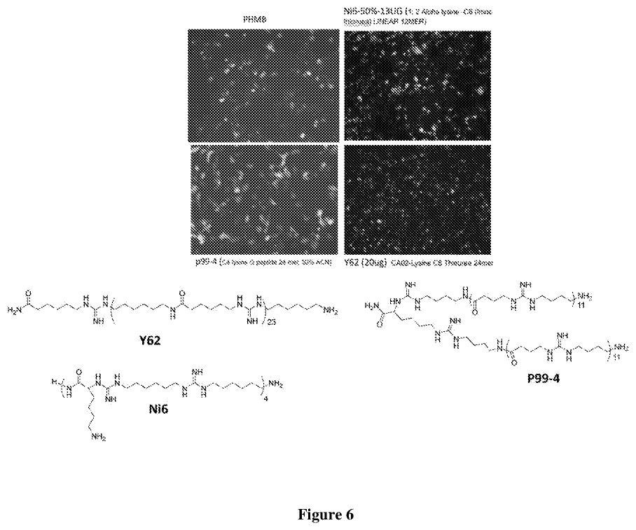

Delivery of pDNA into mammalian cells: To investigate the pDNA carrier potential efficiency of peptides, green fluorescent protein (GFP) expression was measured in HEK cells transfected GFP encoding pDNA-peptide complex using fluorescent microscope. All class of peptides/peptide-oligomers/hybrids of the invention showed pDNA carrier potential, as evident by the expression of GFP in the cells (FIG. 6). Suggesting that the compounds of the present invention not only delivery cargo into cells, and the delivered cargo molecules are released for function (here GFP expression from the pDNA), which could be used to realize various therapeutic potential and also to prepare commercial biosimilar products.

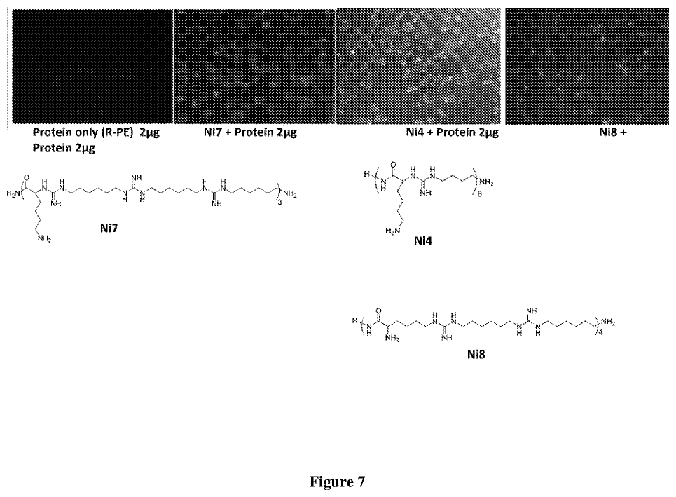

Delivery of proteins into mammalian cells: To check the protein carrying potential of the compound of the invention, we used Vero cells, and transfected them with R-Phycoerythrin (R-PE) protein-peptide complex. Protein delivery potential was confirmed by red fluorescent cells (FIG. 7), suggesting carrier potential of the compounds of the invention.

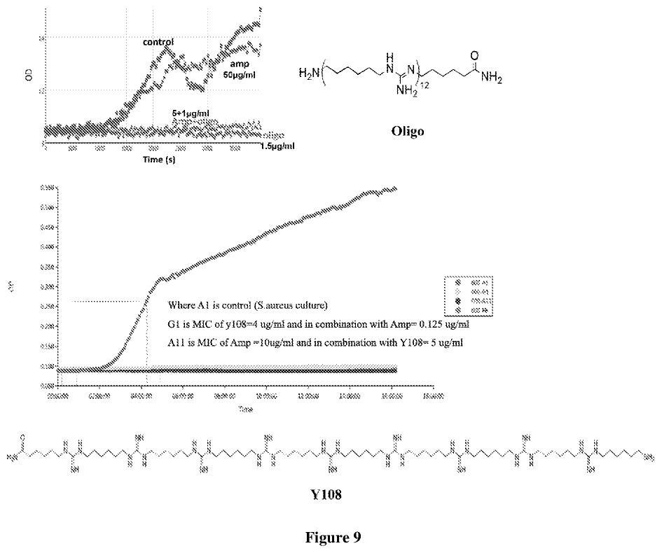

Antibacterial activity of compounds against E. coli and S. aureus: The antibacterial activities of the oligomers/peptides/oligomer-peptide hybrids were investigated using minimum inhibitory concentration (MIC) assays using E. coli or S. aureus. All the compounds tested (oligomers, linear and branched peptides, peptide-oligomer hybrids) showed potent antimicrobial activity against both bacteria, suggesting that the compounds of the invention have antimicrobial activity (FIG. 8). Synergy is evident by a reduction of MIC of ampicillin in presence of oligomer/peptide against resistant S. aureus (FIG. 9), suggesting potentiation of antibiotics.

Antifungal Activity of Peptides Against Saccharomyces cerevisiae and Candida albicans:

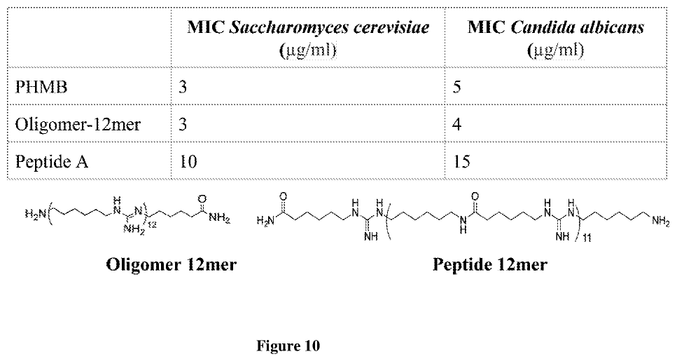

The compounds of the present invention showed potent antifungal activity, as evident by inhibition of growth of Saccharomyces cerevisiae and Candida albicans (FIG. 10). Together, our results suggests that the compounds of the invention have both antibacterial and antifungal activities.

We claim:

1. Cationic guanidine based peptides, oligomers, and oligomer-peptide hybrid compounds obtained from monomer, wherein monomer is represented by Formula 1 below: 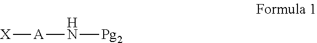
wherein X is 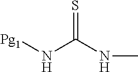
A is selected from the group consisting of -L-, 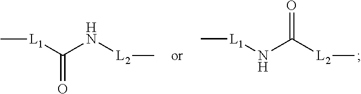
wherein the linker groups, “L, L1, L2,” is an aliphatic group independently selected from propyl (C₃), butyl (C₄), hexyl (C₆), N-substituted alkyl amide, C₁-C₁₄₀ carbon atoms which are selected from group comprising an alkyl group, such as, methylene, ethylene, propylene, C₄, C₅, C₆, C₇, C₈, C₉ or C₁₀; C₁-C₁₀, —C₂₀, —C₃₀, —C₄₀, —C₅₀-C₆₀, —C₇₀, —C₈₀, —C₉₀, —C₁₀₀, —C₁₁₀, —C₁₂₀, —C₁₃₀ or —C₁₄₀, alkyl; cycloaliphatic, heterocyclic, aromatic, aryl, alkylaryl, arylalkyl, oxyalkylene radicals; a polyalkylene radical optionally interrupted by one or more, preferably one, oxygen, nitrogen or sulphur atoms, functional groups, saturated or unsaturated cyclic moiety; or polyethers selected from the group comprising PEG, derivatives of PEG, analogues of PEG;
wherein linker groups “L, L1, L2”, comprise additional cationic moieties selected from the group comprising secondary or tertiary or quaternary amines, or guanidine, or aminoguanidine, diaminoguanidine or biguanidine or analogue, derivative cationic groups either in the backbone of the linker or side chain of the linker, the linker may additionally comprise an amide bond;
wherein Pg1 or Pg2 is independently an orthogonal protecting group selected from the group consisting of Pg1 as Fmoc/Cbz or Boc/Cbz and Pg2 as Boc or Fmoc.

2. The monomer of Formula 1 as claimed in claim 1, consisting of Formula selected from 1a, 1b and 1c: 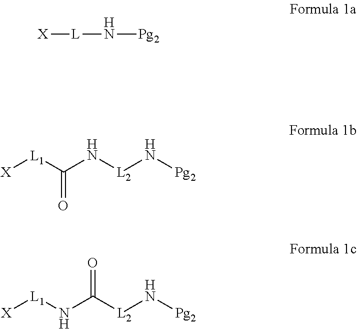
wherein X is 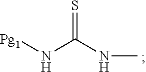
wherein the linker groups “L, L1, L2”, is an aliphatic group containing propyl (C₃), butyl (C₄), hexyl (C₆), N-substituted alkyl amide, C₁-C₁₄₀ carbon atoms selected from group comprising an alkyl group such as methylene, ethylene, propylene, C₄, C₅, C₆, C₇, C₈, C₉ or C₁₀; C₁-C₁₀, —C₂₀, —C₃₀, —C₄₀, —C₅₀-C₆₀, —C₇₀, —C₈₀, —C₉₀, —C₁₀₀, —C₁₁₀, —C₁₂₀, —C₁₃₀ or —C₁₄₀, alkyl; cycloaliphatic, heterocyclic, aromatic, aryl, alkylaryl, arylalkyl, oxyalkylene radicals; a polyalkylene radical optionally interrupted by one or more, preferably one, oxygen, nitrogen or sulphur atoms, functional groups, saturated or unsaturated cyclic moiety; or polyethers selected from the group comprising polyethylene glycol (PEG), derivatives of PEG, analogues of PEG;
wherein linker groups “L, L1, L2” comprise additional cationic moieties selected from the group comprising secondary or tertiary or quaternary amines, or guanidine, or aminoguanidine, diaminoguanidine or analogue, derivative cationic groups either in the backbone of the linker or side chain of the linker, the linker may additionally comprise an amide bond;
wherein Pg1 or Pg2 is independently a protecting group selected from the group consisting of Pg1 as Fmoc/Cbz or Boc/Cbz and Pg2 as Boc or Fmoc.

3. The monomer of Formula 1 as claimed in claim 1, and Formula selected from 1a, 1b and 1c as claimed in claim 2, wherein the monomers are selected from the group comprising:
i. N-(Benzyloxy carbonyl)-N′-(3-tertbutoxycarbonyl amino propylene) thiourea;
ii. N-(Benzyloxy carbonyl)-N′-(4-tertbutoxycarbonyl amino butylene) thiourea;
iii. N-(Benzyloxy carbonyl)-N′-(5-tertbutoxycarbonyl amino pentylene) thiourea;
iv. N-(Benzyloxy carbonyl)-N′-(6-tertbutoxycarbonyl aminohexylene) thiourea;
v. N-(Benzyloxy carbonyl)-N′-(7-tertbutoxycarbonyl amino heptylene) thiourea;
vi. N-(Benzyloxy carbonyl)-N′-(8-tertbutoxycarbonyl amino octylene) thiourea;
vii. N-(-Fluorenyl methoxy carbonyl)-N′-(3-tertbutoxycarbonyl amino propylene) thiourea;
viii. N-(9-Fluorenyl methoxy carbonyl)-N′-(4-tertbutoxycarbonyl amino butylene) thiourea;
ix. N-(9-Fluorenyl methoxy carbonyl)-N′-(5-tertbutoxycarbonyl amino pentylene) thiourea;
x. N-(9-Fluorenyl methoxy carbonyl)-N′-(6-tertbutoxycarbonyl aminohexylene) thiourea;
xi. N-(9-Fluorenyl methoxy carbonyl)-N′-(7-tertbutoxycarbonyl amino heptylene) thiourea;
xii. N-(9-Fluorenyl methoxy carbonyl)-N′-(8-tertbutoxycarbonyl amino octylene) thiourea;
xiii. N-(tertbutoxy carbonyl)-N′-(3-fluorenylmethoxycarbonyl amino propyl) thiourea;
xiv. N-(tertbutoxy carbonyl)-N′-(4-fluorenylmethoxycarbonyl amino butyl) thiourea;
xv. N-(tertbutoxy carbonyl)-N′-(5-fluorenylmethoxycarbonyl amino pentyl) thiourea;
xvi. N-(tertbutoxy carbonyl)-N′-(6-fluorenylmethoxycarbonyl amino hexyl) thiourea;
xvii. N-(tertbutoxy carbonyl)-N′-(7-fluorenylmethoxycarbonyl amino heptyl) thiourea;
xviii. N-(tertbutoxy carbonyl)-N′-(8-fluorenylmethoxycarbonyl amino octyl) thiourea;
xix. 4-fluorenylmethoxycarbonyl amino-N-(4-(3-tertbutyloxycarbonyl thioureido)butyl) butanamide;
xx. 3-fluorenylmethoxycarbonyl amino-N-(4-(3-tertbutyloxycarbonyl thioureido)butyl) propanamide;
xxi. 3-fluorenylmethoxycarbonyl amino-N-(5-(3-tertbutyloxycarbonyl thioureido) pentyl) propanamide;
xxii. 3-fluorenylmethoxycarbonyl amino-N-(6-(3-tertbutyloxycarbonyl thioureido) hexyl) propanamide;
xxiii. 6-fluorenylmethoxycarbonyl amino-N-(6-(3-tertbutyloxycarbonyl thioureido) hexyl) hexanamide;
xxiv. 4-fluorenylmethoxycarbonyl amino-N-(5-(3-tertbutyloxycarbonyl thioureido) pentyl) butanamide;
xxv. 4-fluorenylmethoxycarbonyl amino-N-(6-(3-tertbutyloxycarbonyl thioureido) hexyl) butanamide;
xxvi. 6-tertbutyloxycarbonyl amino-N-(6-(3-fluorenylmethoxycarbonyl thioureido) hexyl) hexanamide;
xxvii. 6-(3-fluorenylmethoxycarbonyl thioureido)-N-(6-tertbutyloxycarbonylamino) hexyl) hexanamide.

4. Cationic guanidine based peptides, oligomers, and oligomer-peptide hybrid compounds, obtained from monomer of Formula 1a as claimed in claim 2, represented by Formula 2: 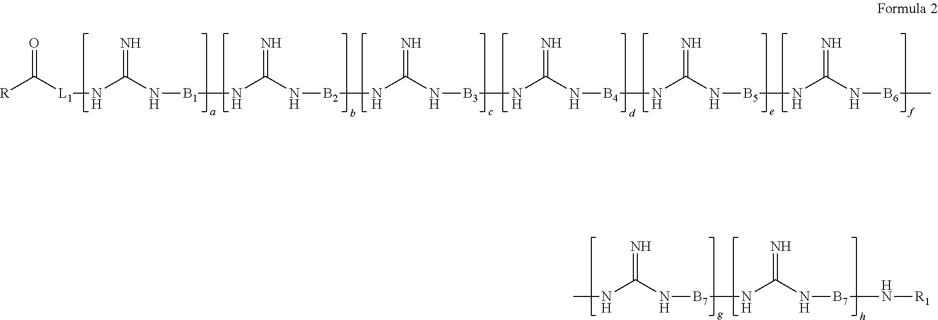 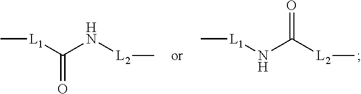
wherein “B1-B7” is independently selected from the L or 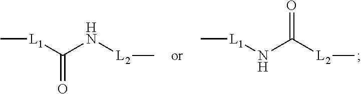
or combination of L and where R is independently selected from selected from the group consisting of NH₂, OH;
where L1, L2, L3 are selected from the group consisting of C₂-C₈; 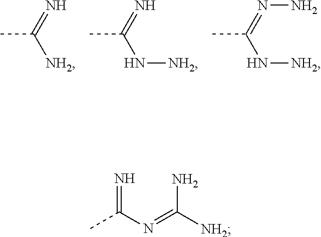
where-R1 is independently selected from —H, -acetyl,
and where R1 is defined in such a way that it yields guanidine, aminoguanidine, biguanidine, diaminoguanidine; and in addition R1 is added by linkage to aliphatic, cycloaliphatic heterocyclic, heterocyclic, aryl, alkylaryl, arylalkyl, oxyalkylene radicals;
and in addition end groups may be modified by linkage to receptor ligands, dextrans, cyclodextrins, fatty acids or fatty acid derivatives, lipids, cholesterol or cholesterol derivatives or polyethylene glycol (PEG);
where a, b, c, d, e, f, g, h each independently selected from any integer chosen from 0 to 100, for example, from 0, 1, 2, 3, 4 or 5 to 10, 20, 30, 40, 50, 60, 70, 80, 90, 100, where the sum of a-h is at least 1.

5. Compounds of Formula 2 as claimed in claim 4, include but are not limited to:

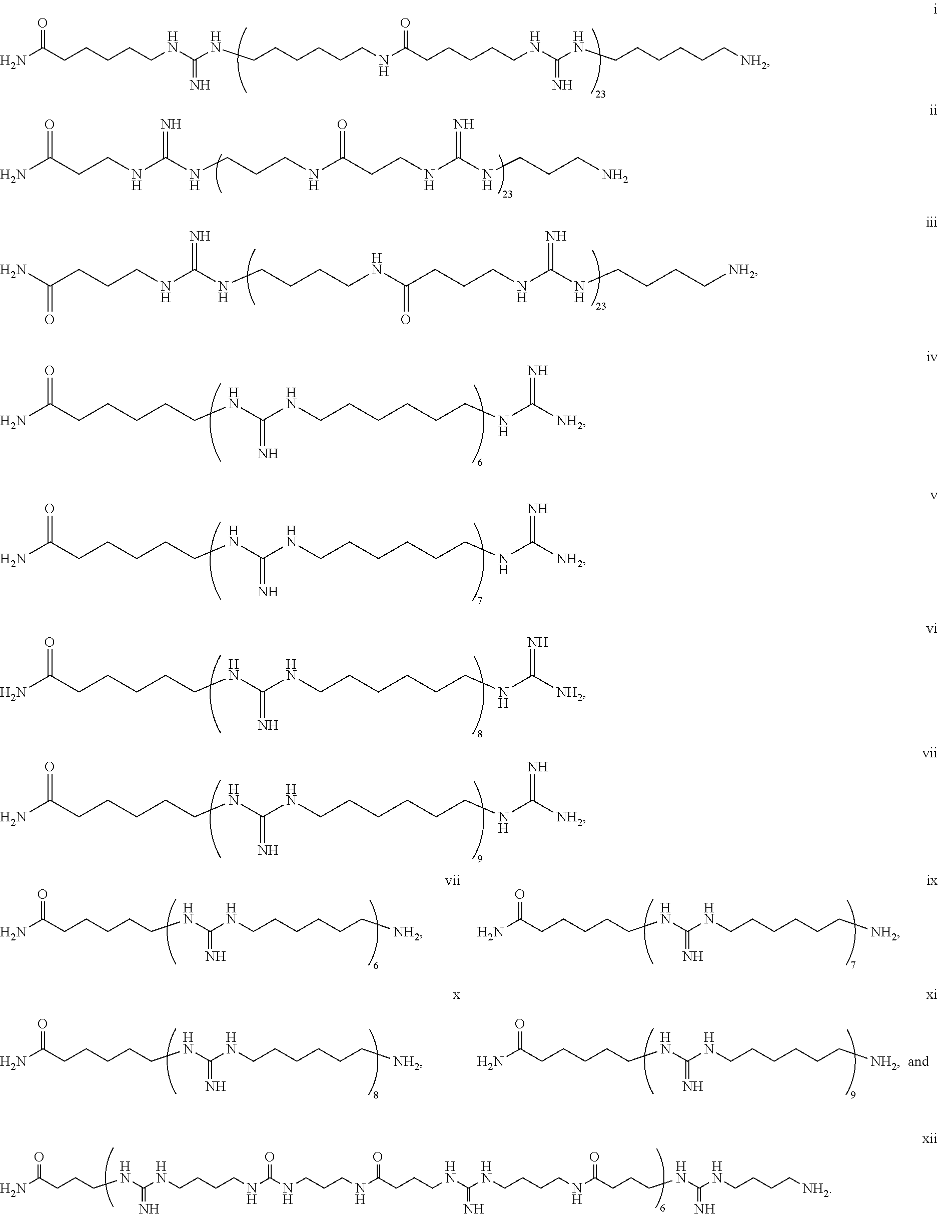

6. Oligoguanidine compounds of Formula 2 as claimed in claim 4, include but are not limited to 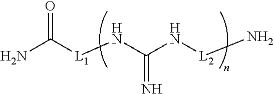
wherein L₁=—(CH₂)₅, L₂=—(CH₂)₆, n=2-30,
or wherein L₁=—(CH₂)₃, L₂=—(CH₂)₄, n=2-30,
or wherein L₁=—(CH₂)₂, L₂=—(CH₂)₃, n=2-30,
or 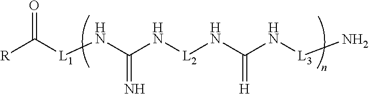
wherein L₁=—(CH₂)₅, L₂=—(CH₂)₆, L₃=—(CH₂)₃, n=1-30,
or wherein L₁=—(CH₂)₅, L₂=—(CH₂)₆, L₃=—(CH₂)₄, n=1-30,
or wherein L₁=—(CH₂)₅, L₂=—(CH₂)₃, L₃=—(CH₂)₄, n=1-30,
guanidine backbone peptides of Formula 2 as claimed in claim 4, include but are not limited to 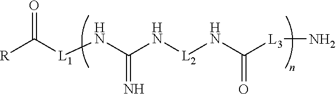
wherein L₁=—(CH₂)₅, L₂=—(CH₂)₆, L₃=—(CH₂)₅, n=2-30,
or wherein L₁=—(CH₂)₃, L₂=—(CH₂)₄, L₃=—(CH₂)₃, n=2-30,
or wherein L₁=—(CH₂)₂, L₂=—(CH₂)₃, L₃=—(CH₂)₂, n=2-30,
or wherein L₁=—(CH₂)₅, L₂=—(CH₂)₆, L₃=—(CH₂)₂, n=2-30,
or wherein L₁=—(CH₂)₅, L₂=—(CH₂)₆, L₃=—(CH₂)₃, n=2-30,
or wherein L₁=—(CH₂)₅, L₂=—(CH₂)₄, L₃=—(CH₂)₂, n=2-30.

7. Cationic guanidine based oligomer-peptide hybrid compound, obtained from monomer of Formulae 1a, 1b and 1c as claimed in claim 2, represented by Formula 3 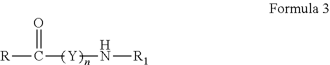
wherein Y is selected from
-(AA1)ₘ₁-(AA2)ₘ₂-(AA3)ₘ₃-(AA4)ₘ₄-(AA5)ₘ₅-(AA6)ₘ₆-(OG1)ₙ₁-(OG2)ₙ₂-(OG3)ₙ₃-(OG4)ₙ₄-(OG5)ₙ₅-(OG6)ₙ₆-,
-(OG1)ₙ₁-(OG2) 2-(OG3)ₙ₃-(OG4)ₙ₄-(OG5)ₙ₅-(OG6)ₙ₆-(AA1)ₘ₁-(AA2)ₘ₂-(AA3)ₘ₃--(AA4)ₘ₄-(AA5)ₘ₅-(AA6)ₘ₆-,
-(AA1)ₘ₁-(OG1)ₙ₁-(AA2)ₘ₂-(OG2)ₙ₂-(AA3)ₘ₃-(OG3)ₙ₃-(AA4)ₘ₄-(OG4)ₙ₄-(AA5)ₘ₅-(OG5)ₙ₅-(AA6)ₘ₆-(OG6)ₙ₆-,
-(AA1)ₘ₁-(OG)ₙ₁, -(OG1)ₘ₁-(AA1)ₙ₁-; -(OG1)ₘ₁-(AA1)ₙ₁-(OG2)ₘ₂-; or -(AA1)ₘ₁-(OG1)ₙ₁-(AA2)ₘ₂-
wherein
m1, m2, m3, m4, m4, m6, n1, n2, n3, n4, n5, n6 are integers independently selected from 0, 1, 2 . . . 100 and n is an integer independently selected from 1 to 100; m1, n1, m2 and n2 is similar or different; where the sum of m1-m6 and n1-n6 is at least 2;
R together with the carbonyl group to which it is attached is independently selected from —H, free acid, amide, amine, acid salt, ester or functionalized carbonyl group;
R1 is independently selected from —H, -acetyl, 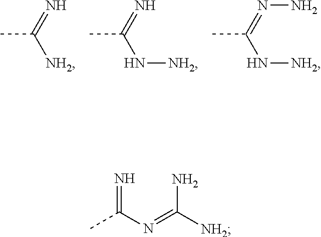
R1 is designed such a way that it yields guanidine, aminoguanidine, biguanidine, diaminoguanidine, additionally, R1 is added by linkage to aliphatic, cycloaliphatic heterocyclic, heterocyclic, aryl, alkylaryl, arylalkyl, oxyalkylene radicals; wherein end groups may be modified by linkage to receptor ligands, dextrans, cyclodextrins, fatty acids or fatty acid derivatives, lipids, cholesterol or cholesterol derivatives or polyethylene glycol (PEG);
and
OG1, OG2, OG3, OG4, OG5, OG6 is independently selected from the oligomer residue or guanidine backbone amino acid residue arising from the monomer of Formula 1a or Formulae 1b and 1c, respectively;
AA1, AA2, AA3, AA4, AA5, AA6 is independently selected from amino acids residue chosen from any of the natural amino acid residues (D and L form)/synthetic amino acid, such that the AA1 (amino acid residue) is present at any position in the oligomer, for example at C-terminus, N-terminus, in between guanidine group of the oligomer residue.

8. The oligomer of formulae 2 and 3 as claimed in claim 4 and claim 6, respectively, is in the form of a linear chain, branched chain, dendrimer forms or cyclic structure.

9. The compounds of formula 3 as claimed in claim 6 include, but are not limited to:

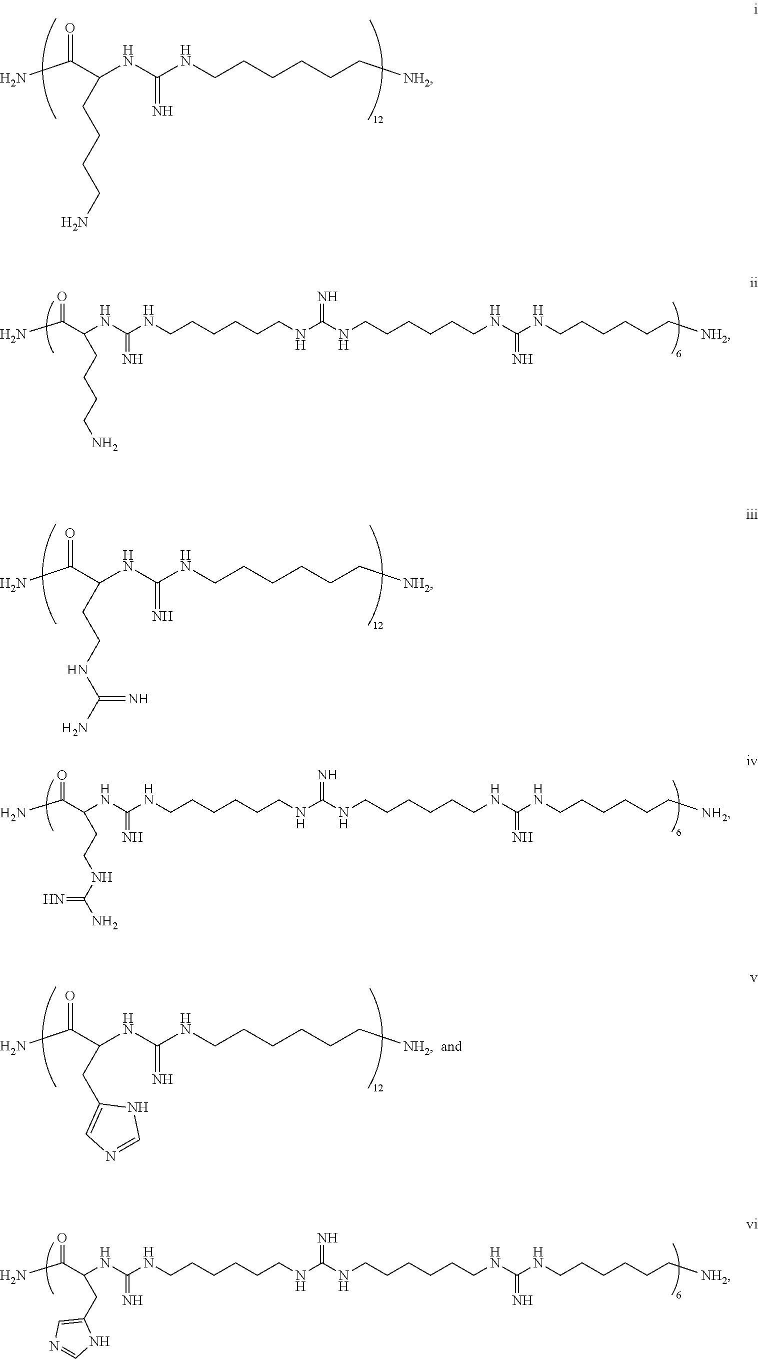

10. Peptide-oligomer hybrids of Formula 3 as claimed in claim 6 include, but are not limited to: 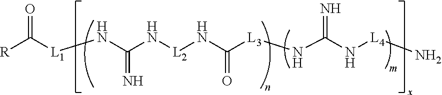
wherein L₁=—(CH₂)₅, L₂=—(CH₂)₆, L₃=—(CH₂)₅, L₄=—(CH₂)₆, m=2-30, n=2-30, x=2-30
or wherein L₁=—(CH₂)₃, L₂=—(CH₂)₄, L₃=—(CH₂)₃, L₄=—(CH₂)₄, m=2-30, n=2-30, x=2-30
or wherein L₁=—(CH₂)₂, L₂=—(CH₂)₃, L₃=—(CH₂)₂, L₄=—(CH₂)₃, m=2-30, n=2-30, x=2-30
or wherein L₁=—(CH₂)₅, L₂=—(CH₂)₆, L₃=—(CH₂)₂, L₄=—(CH₂)₆, m=2-30, n=2-30, x=2-30
or wherein L₁=—(CH₂)₅, L₂=—(CH₂)₆, L₃=—(CH₂)₃, L₄=—(CH₂)₆, m=2-30, n=2-30, x=2-30
or wherein L₁=—(CH₂)₃, L₂=—(CH₂)₄, L₃=—(CH₂)₂, L₄=—(CH₂)₄, m=2-30, n=2-30, x=2-30
or wherein L₁=—(CH₂)₂, L₂=—(CH₂)₃, L₃=—(CH₂)₃, L₄=—(CH₂)₃, m=2-30, n=2-30, x=2-30.

11. A process for preparing the compounds of Formula 1a as claimed in claim 2, said process comprising the steps of:
i. treating a diamine compound with di-tertbutyl carbonate orthogonal amine protecting group in chloroform as a solvent at 0-10° C., for 12-24 hours to provide intermediate (A) wherein one of the diamine is selectively protected; and
ii. treating the intermediate A obtained in step (i) with protected isothiocyanate (B) in tetrahydrofuran (THF) solvent at 4-25° C., for 12-24 hours to obtain the compound of Formula 1a

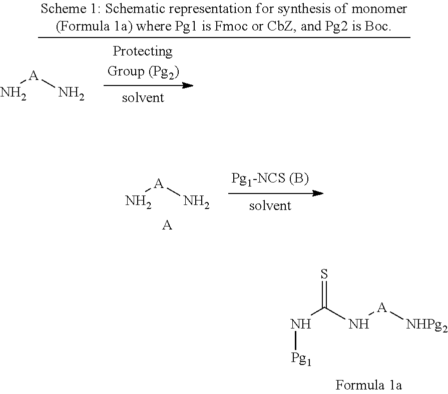

12. The process as claimed in claim 11, wherein the diamine compound is present at 10 equiv, di-tertbutyl carbonate orthogonal amine protecting group is present in 1 equiv and the intermediate A in step ii is present at 1 equiv.

13. A process for preparing the compounds of Formula 1a as claimed in claim 2, said process comprising the steps of:
i. To a precooled 0-10° C. solution of 1,6-diamino hexane (10 equiv) in 100 volumes of non-polar solvent (chloroform), di-tertbutyl carbonate (1 equiv) in chloroform (10 volume) was added dropwise and stirred for 12-24 hours; after completion of reaction the intermediate (A) was isolated in 60-80% yield wherein one of the diamines is selectively protected; and
ii. To the solution of intermediate A in non-polar solvent (tetrahydrofuran, 10 volume) obtained in step i, 1 equiv, of fluorenylmethoxycarbonyl isothiocyanate (Fmoc-NCS) was added and stirred for 0.5-3 hours at 4-25° C.; after completion of reaction as monitored over TLC; hexane (non-polar solvent 200 volume) was added and stirred to obtain the precipitated compound of Formula 1 in 50-70% yield; in a similar manner, benzyloxycarbonyl isothiocyanate (Cbz-NCS) has also been used to synthesize another form of Formula 1;

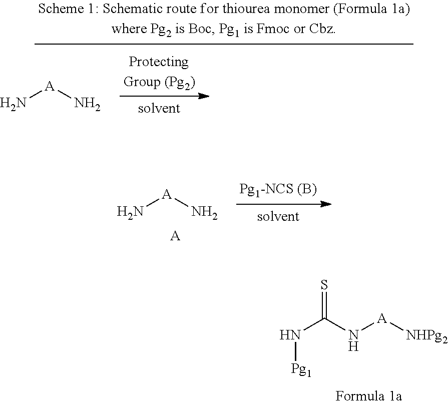

14. The process as claimed in claim 13, wherein 1,6-diamino hexane is present at 10 equiv in 100 volumes of non-polar solvent which is chloroform, di-tertbutyl carbonate is present at 1 equiv in chloroform in 10 volume in step i; and in step ii, the non-polar solvent is tetrahydrofuran in 10 volume, 1 equiv of fluorenylmethoxycarbonyl isothiocyanate (Fmoc-NCS) and non-polar solvent hexane is present in 200 volume.

15. A process for preparing the compounds of Formula 1a as claimed in claim 2, said process comprising the steps of:
i. the tert-butylcarbamate (Boc-amine, 1 equiv) was treated with carbon disulfide (5-10 equiv, CS₂) in presence of KOH (1 equiv) using hexane:ethylacetate (2:1) solvent mixture at 10-25° C. for 12-24 hours to form potassium tert-butylcarbamodithioate; then, tert-butylcarbamodithioate was treated with sodium persulphate (1 equiv) in aqueous media with ice cold condition for 10-30 min and completion of reaction was monitored with TLC; the tert-butyloxycarbonyl isothiocyanate (Boc-NCS) was extracted with dichloromethane and used in next step reaction without isolation;
ii. second method to synthesize Boc-NCS from tert-butylcarbamodithioate in organic solvent was also employed; in brief, tert-butyl carbamodithioate (1 equiv) was suspended in THF (10 volume) and 0.9 equiv, of Boc-anhydride was added in ice cold condition and reaction mixture was stirred for 15 min; then, solution of (0.1-0.3 equiv) 4-dimethylamino pyridine (DMAP) in THF was added and left for stirring for another 15 min; the reaction mixture was filtered and used for the next step immediately;
iii. the solution of 1,6-diamino hexane (3 equiv) in DCM was treated Boc-NCS (dropwise addition) at 4-25° C. to yield in 30-50% of 1-(6-aminohexyl)-3-(tert-butyloxycarbonyl) thiourea (A); then, further free amine of thiourea (A) was reacted with Fmoc-OSu (1.1 equiv) and after completion of reaction as monitored over TLC, the solvent was evaporated and reconstituted in nonpolar solvent (DCM) and organic phase was treated with 10% citric acid followed by distilled water and brine solution; The organic phase was dried over sodium sulphate and compound was isolated after evaporating the organic phase; The compound was purified over flash chromatography 30% ethylacetate: Hexane (30-50% yield); the synthesize monomer (Formula 1a) having Fmoc as Pg₁ and Boc as Pg₂

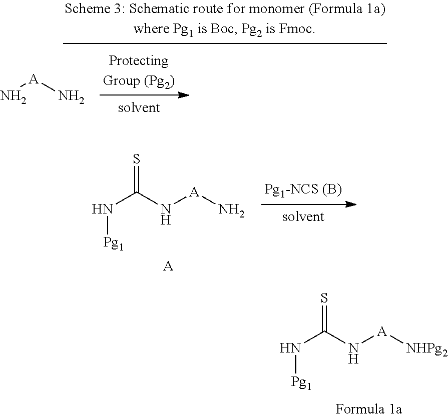

16. A process for preparing the compounds of Formulae 1b and 1c as claimed in claim 2, said process comprising the steps of:
i. for compound with Formula 1b with amide bond, N-Fmoc-6-amino hexanoic acid (1 equiv) in nonpolar solvent (DCM, 10 volume) was treated with 1.1 equiv, of 1-Ethyl-3-[3-dimethylaminopropyl] carbodiimide hydrochloride (EDC or EDAC) and 1.1 equiv, of HOBt for 1-3 hours at 10-30° C.; the resulting activated carboxylic acid group was reacted with N-boc-1,6 diamine at 20-30° C. for 12-24 hours; after completion of reaction as monitored over TLC, the organic phase was washed with 10% citric acid followed by 10% sodium bicarbonate, distilled water and brine solution; the organic phase was dried over sodium sulphate and evaporated to obtain the compound (A) in 50-70% yield; further compound was treated with 20% piperidine in DMF for 1-4 hours to deprotect the fmoc group; after completion of the reaction, the compound was precipitated by adding water and filtered, dried; the solution of deprotected compound in non-polar solvent (THF), Fmoc-NCS (1 equiv) was added and stirred for 0.5-2 hours at 20-30° C.; the compound was precipitated by addition of hexane (200 volume) and filtered to yield compound with Formula 1b in 30-50% yield; 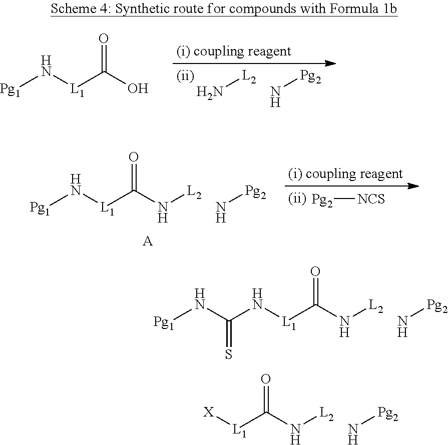
ii. for synthesis of compounds with Formula 1c, the compound A from Scheme 4 was treated with DCM;TFA (1:1) to deprotect Pg2 group (Boc) and precipitated in diethylether, the obtained compound (1 equiv) was reacted with Boc-NCS (1 equiv) in presence of organic base (such as, trimethylamine, diisopropyl ethyl amine, 3 equiv, 4-30° C., 0.5-3 hours) to yield the compound with Formula 1c; alternatively, N-Boc-6-amino hexanoic acid (1 equiv) in nonpolar solvent (DCM, 10 volume) was treated with 1.1 equiv; of 1-Ethyl-3-[3-dimethylaminopropyl] carbodiimide hydrochloride (EDC or EDAC) and 1.1 equiv, of HOBt for 1-3 hours at 10-30° C.; the resulting activated carboxylic acid group was reacted with 1,6 diamine (3 equiv) at 20-30° C. for 12-24 hours; after completion of reaction as monitored over TLC, the organic phase was washed with distilled water and brine solution; the organic phase was dried over sodium sulphate and evaporated to obtain the compound (A) in 50-70% yield; the compound C (1 equiv, in THF) was treated with Fmoc-NCS (1 equiv) for 0.5-2 hours at 20-30° C.; after completion of the reaction, the final compound with Formula 1c was precipitated by addition of hexane (200 volume) and filtered, dried in 30-50% yield;

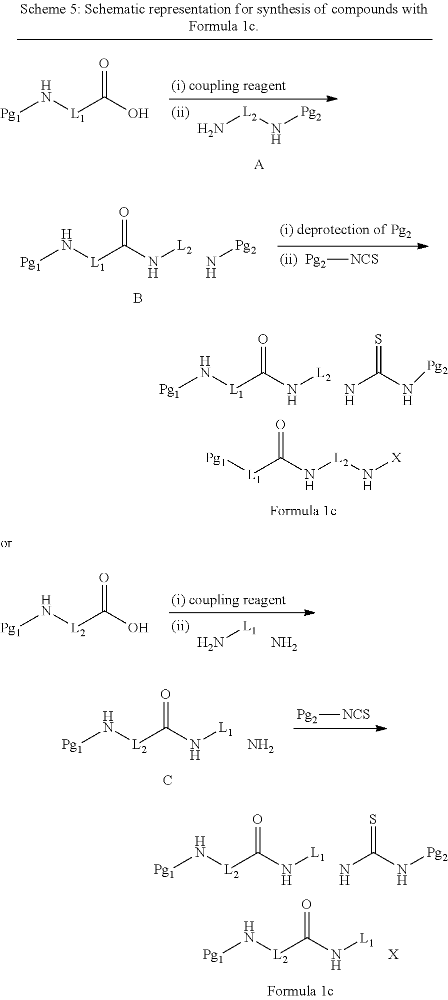

17. A process to make oligomer of Formula 2 as claimed in claim 4 in a controlled manner as shown in Scheme 6, such that the obtained oligomer is pure and free from side products suitable for pharmaceutical applications, and the process includes the steps of:
i. placing MBHA (Methylbenzhydrylamine) resin in a peptide synthesizer vessel; Wang resin, Rink amide MBHA resins can also be used;
ii. swelling the resin with suitable solvent (DCM) followed by washing with solvent(s);
iii. carboxylic acid coupling method (coupling method A) wherein the resin is treated with acid reactant (C) having Fmoc as protected group using HBTU (coupling agent) and DIPEA (a base) in DMF (solvent) to provide intermediate (D); other protecting groups, for example, Boc are used;
iv. capping of free amines on resin-optional capping of free amino groups using acetic anhydride in DMF (a solvent);
v. deprotection method wherein deprotection of intermediate (D) using 20% piperidine (for Fmoc)/50% TFA-5% m-cresol-45% DCM (for Boc) followed by washing with DMF, DCM or their mixture and last washing with DCM;
vi. coupling for monomer (Formula 1) (coupling method B) wherein the monomer (Formula 1) was added to the mixture of 12/TMP (1:3 equiv) or N-iodosuccinimide (NIS) or 1,3-Diiodo-5,5-Dimethylhydantoin (DIH) or any other desulphurizing agent in a solvent medium and the free amine containing resin is treated to form the coupled product (E);
vii. deprotection of coupled product (E) followed by washing and then reaction with second unit of monomer of Formula 1 as claimed in claim 1
viii. repetition of step F until the oligomer with desired number of repeating units is formed; and
ix. cleaving of the resin followed by precipitation and desalted using C18 silica and lyophilized using temperature (−20° C. to −110° C.) and pressure (range of 0.01 to 1.0 mbar) to obtain the compound of Formula 2 as claimed in claim 4

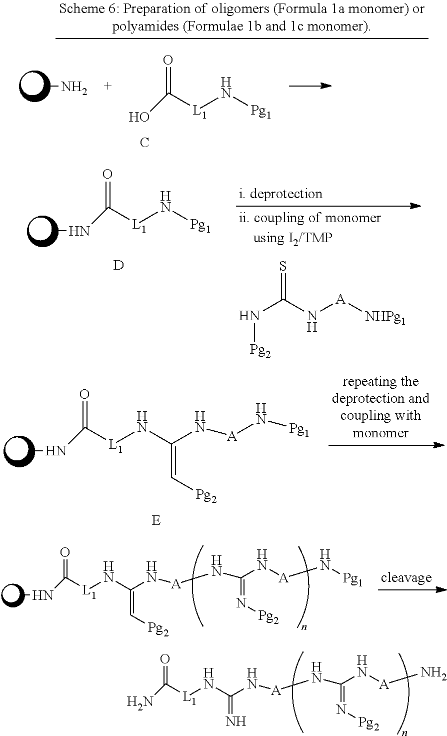

18. A process to obtain the oligomer-peptide hybrid compound of Formula 3 as claimed in claim 6 in a controlled way, said process includes the steps of:
i. treating MBHA resin (A) with acid reactant (B) in the presence of coupling agent and a base in a solvent to the corresponding amide followed by deprotection;
ii. reacting intermediate from step i with monomer of Formula 1 using TMP/I₂ followed by deprotection to give guanidine-based amine intermediate C;
iii. acid-amine coupling of guanidine based intermediate C with amine-protected amino-acid, followed by deprotection to obtain coupled product D;
iv. reacting coupled product D with second unit of monomer of Formula 1 using TMP/I₂, deprotection and then again reaction with 3^{rd} unit of monomer of Formula 1 using TMP/I₂ and deprotection;
v. continuation of reaction until introduction of nth unit of monomer of Formula 1 and final deprotection and resin cleavage;
vi. sequence of coupling with amino-acid or monomer and the number of amino-acid residue and monomer unit can vary according to the targeted structure of the oligomer-peptide hybrid compound;

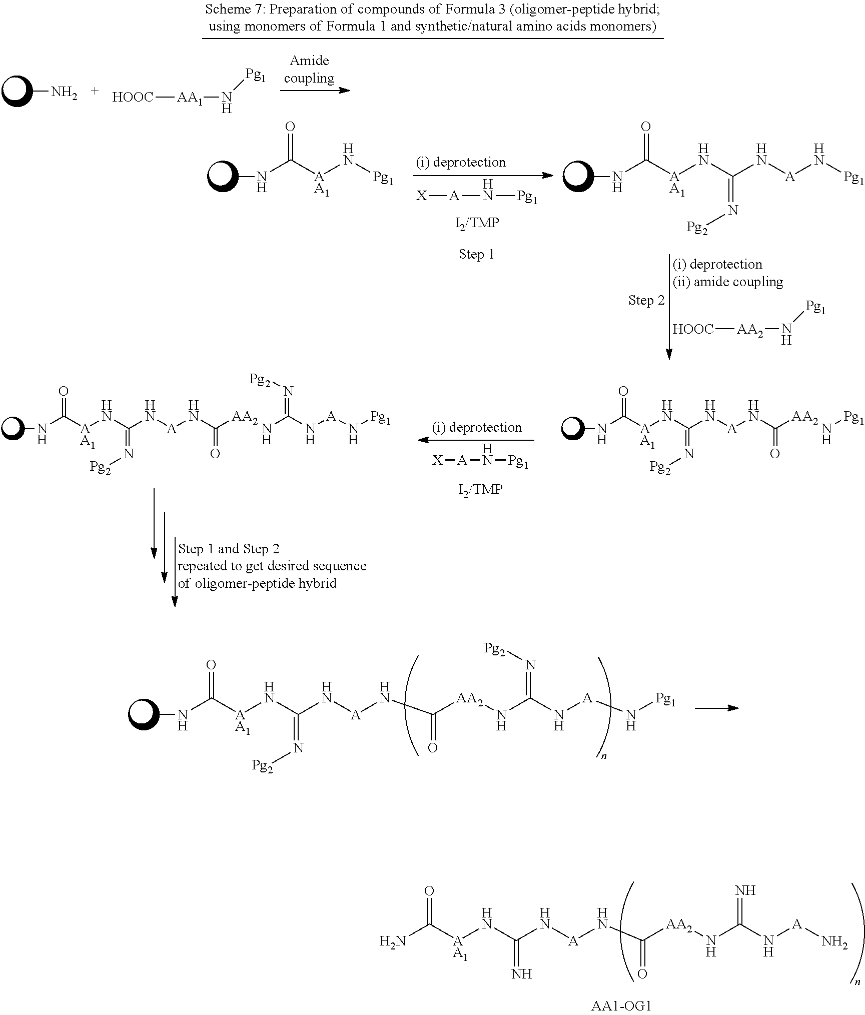

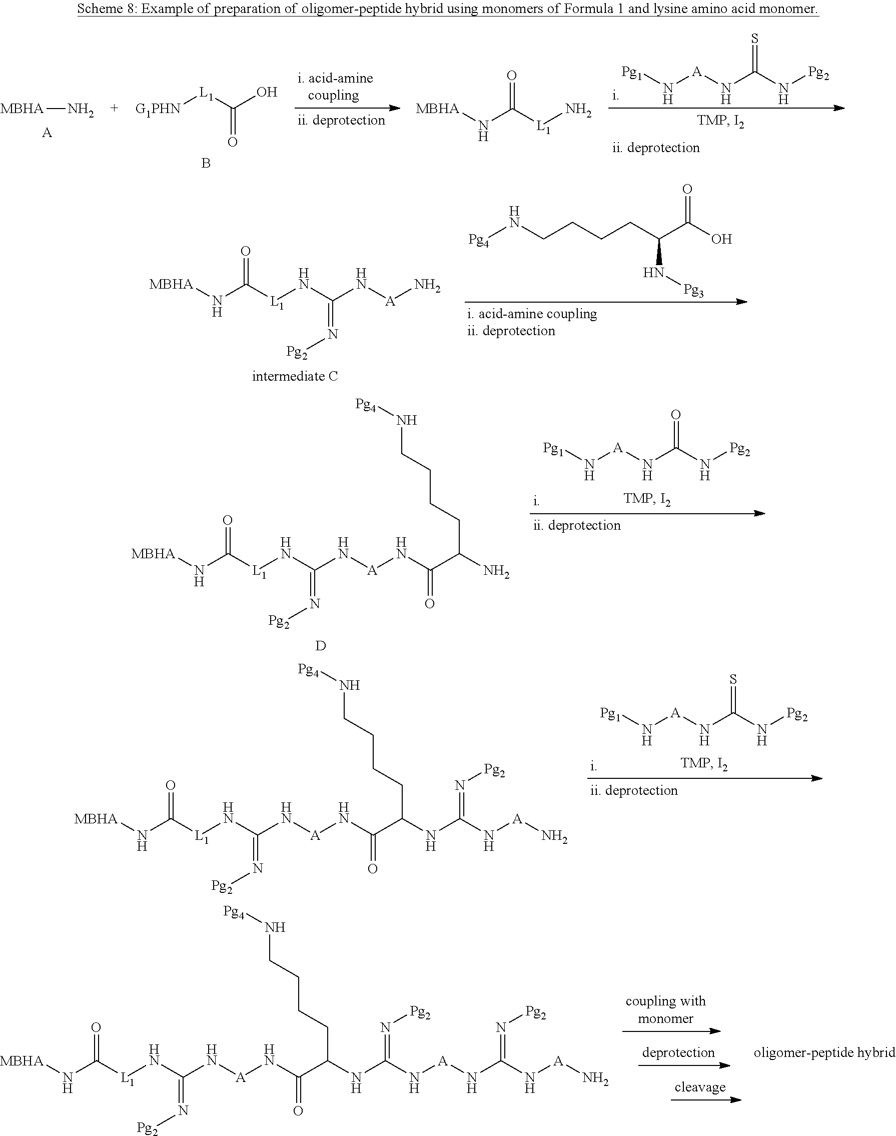

19. A process to produce cationic backbone peptides using monomers of Formulae 1b and 1c as claimed in claim 2, said process includes the steps of:
i. treating MBHA resin with acid reactant (C) in the presence of coupling agent and a base in a solvent to the corresponding amide followed by deprotection to obtain an intermediate D;
ii. reacting intermediate D after deprotection with monomer of Formula 1b or 1c using TMP/I₂ to give guanidine-based amine intermediate E;
iii. the deprotection of Pg1 followed by coupling of formula 1b or 1c using TMP/I₂, the same process repeated until desired length of the compound being made; then final cleavage of the compound, precipitation and followed by desalting to yield purified compound;

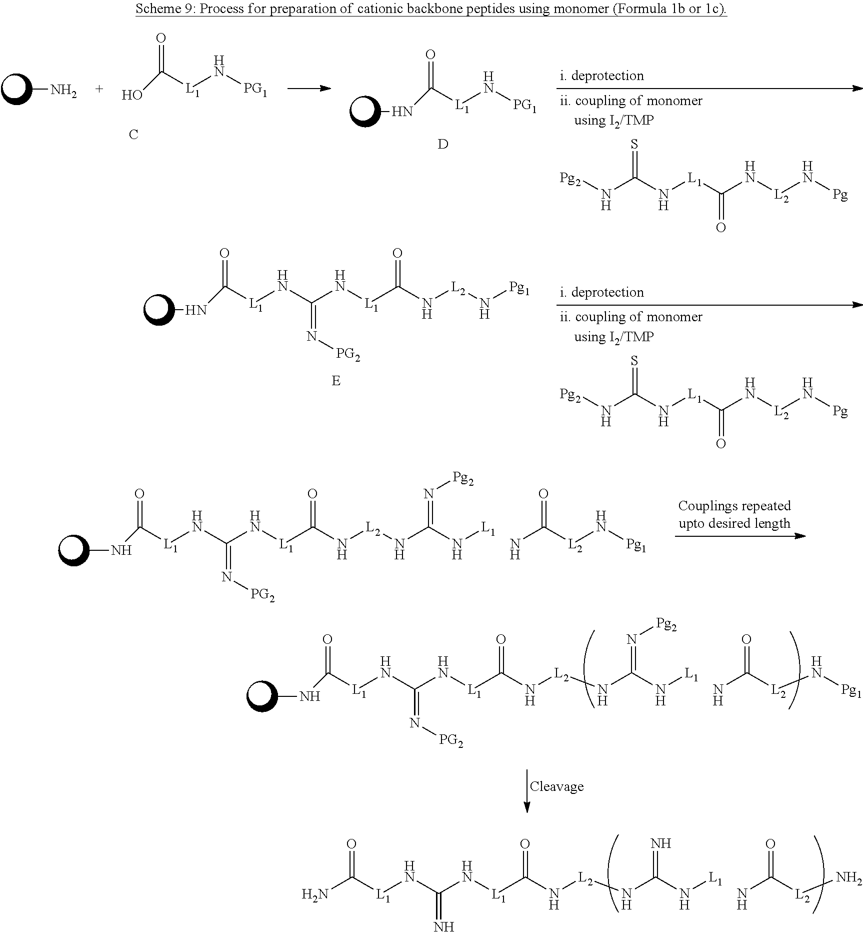

20. A process to produce cationic backbone peptides using monomers of Formula 1a, as claimed in claim 2, said process comprising steps:
i. treating MBHA resin with acid reactant (C) in the presence of coupling agent and a base in a solvent to the corresponding amide followed by deprotection to obtain an intermediate D;
ii. reacting intermediate D after deprotection with monomer of Formula 1a using TMP/I₂ to give guanidine-based amine intermediate E;
iii. the intermediate E after deprotection reacted with amine protected unnatural amino acid (F) in presence of HBTU (coupling reagent) and DIPEA (a base);
iv. then again, deprotection followed by coupling of G using TMP/I₂ to give guanidine;
v. The amide and thiourea couplings were proceeded to yield guanidine-polyamide using Formula 1a;

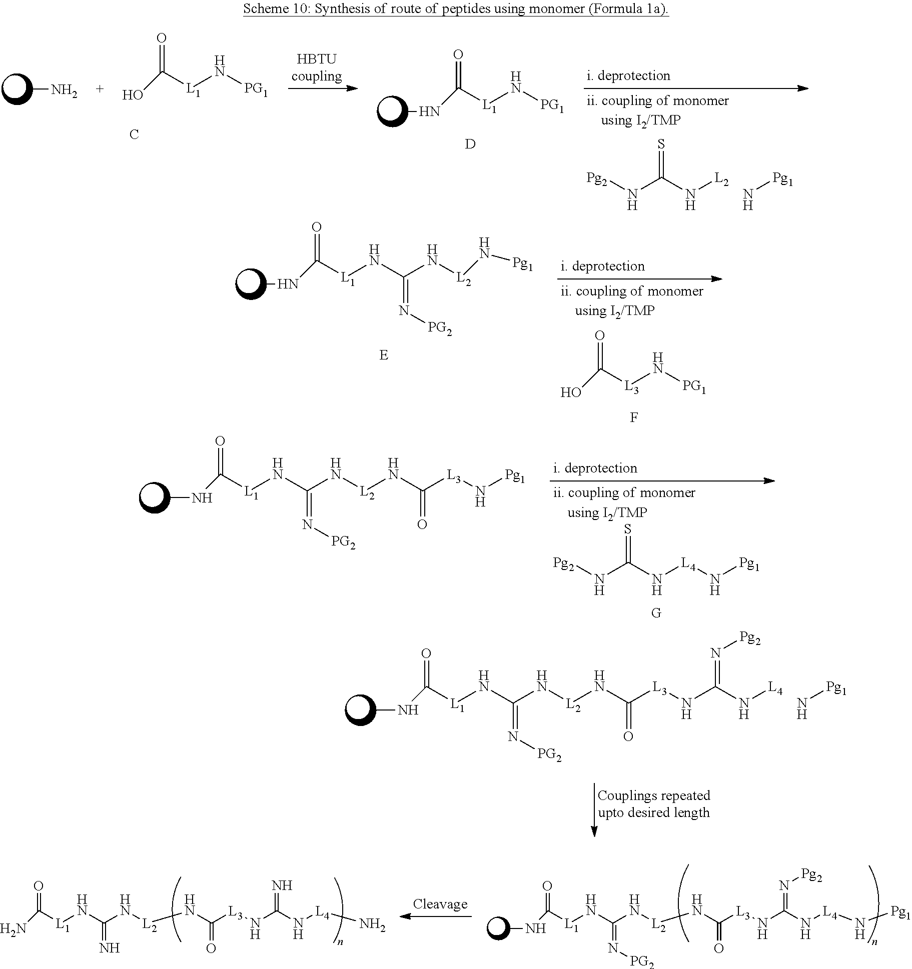

21. A pharmaceutical composition comprising the peptide, oligomer, oligomer-peptide hybrid compounds as claimed in claims 1, 4 and 7 along with pharmaceutically acceptable excipients.

22. The pharmaceutical composition as claimed in claim 21 when administered orally or parenterally or topically.

23. The compound or composition as claimed in claim 1, 4, or 7 for its utility as antimicrobial, antifungal agent.

24. The compound or composition as claimed in claim 1, 4, or 7 for its utility as a carrier for other molecules into eukaryote and prokaryote cells, in-vitro, ex-vivo and in-vivo; the said compound is used at 0.01-1000 fold molar or weight/weight excess over introduced agents (nucleic acids/analogues, peptide/proteins/analogues, small molecules drugs including antibacterial and antifungals).

25. The composition as claimed in claim 21 in the form of liquid injectables or oral dosage form, comprising (tablets, or capsules, solutions or suspensions), or topically in the form of ointment, cream, spray, bandages or powder, alternatively or as intramammary preparations, wherein the compound is the dose of 0.1 to 100 mg/Kg body weight of the mammal.
